(19) United States
(12) Patent Application Publication
(10) Pub. No.: US 2024/0368457 A1
(43) Pub. Date: Nov. 7, 2024

(54) ULTRA SHORT HYDROPHOBES FOR UNCONVENTIONAL RESERVOIRS WITH LIGHT OILS CONTAINING SOLUTION GAS

(71) Applicants: ULTIMATE EOR SERVICES LLC, Austin, TX (US); HARCROS CHEMICALS, INC., Kansas City, KS (US)

(72) Inventors: Upali WEERASOORIYA, Austin, TX (US); Don Pathma Jith LIYANAGE, Pflugerville, TX (US); Karasinghe Arachchige Nadeeka UPAMALI, Austin, TX (US); Christopher James BRITTON, Leander, TX (US); Robert Matthew DEAN, Austin, TX (US); John BOOREM, Kansas City, KS (US); Kurt CHESHIRE, Shawnee, KS (US)

(21) Appl. No.: 18/687,037

(22) PCT Filed: Aug. 29, 2022
(86) PCT No.: PCT/US2022/041941

§ 371 Date: Feb. 27, 2024

Related U.S. Application Data
(60) Provisional application No. 63/237,907, filed on Aug. 27, 2021.

(51) Int. Cl. C09K 8/588; C08G 65/34; C08J 3/03; C09K 8/584; E21B 43/16

(52) U.S. Cl. CPC C09K 8/588; C08G 65/34; C08J 3/03; C09K 8/584; E21B 43/16

(57) ABSTRACT

Disclosed are compounds as well as methods of using thereof in oil and gas operations. The compounds described herein can be used in Enhanced Oil Recovery (EOR) formulations to provide aqueous stability and ultra-low interfacial tension region. Accordingly, also provided are aqueous compositions for use in EOR that comprise the compounds described herein.

### CROSS-REFERENCE TO RELATED APPLICATIONS

This application claims benefit of U.S. Provisional Application No. 63/237,907, filed Aug. 27, 2021, which is hereby incorporated herein by reference in its entirety.

### BACKGROUND

Enhanced Oil Recovery (EOR) refers to techniques for increasing the amount of unrefined petroleum, or crude oil that may be extracted from an oil reservoir (e.g., an oil field). Using EOR, 40-60% of the reservoir's original oil can typically be extracted compared with only 20-40% using primary and secondary recovery (e.g., by water injection or natural gas injection). Enhanced oil recovery may also be referred to as improved oil recovery or tertiary oil recovery (as opposed to primary and secondary oil recovery).

Enhanced oil recovery may be achieved by a variety of methods including miscible gas injection (which includes carbon dioxide flooding), chemical injection (which includes polymer flooding, alkaline flooding, and surfactant flooding), microbial injection, or thermal recovery (which includes cyclic steam, steam flooding, and fire flooding). The injection of various chemicals, usually as dilute aqueous solutions, has been used to improve oil recovery. Injection of alkaline or caustic solutions into reservoirs with oil that has organic acids naturally occurring in the oil (also referred to herein as “unrefined petroleum acids”) will result in the production of soap that may lower the interfacial tension enough to increase production. Injection of a dilute solution of a water soluble polymer to increase the viscosity of the injected water can increase the amount of oil recovered from geological formations. Aqueous solutions of surfactants such as petroleum sulfonates may be injected to lower the interfacial tension or capillary pressure that impedes oil droplets from moving through a reservoir. Special formulations of oil, water and surfactant microemulsions have also proven useful. Such formulations often include co-solvent compounds to increase the solubility of the solutes in the presence of oil and decrease the viscosity of an emulsion.

There is a need in the art for cost effective methods for enhanced oil recovery using chemical injection. Provided herein are methods and compositions addressing these and other needs in the art.

### SUMMARY

Provided herein are compounds, compositions, and methods for enhanced oil recovery. A compound described herein can be defined by Formula I

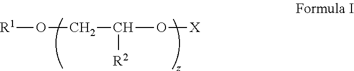

wherein
R¹ is a C₁-C₈ alkyl group or C₆ aryl group;
R² is, independently for each occurrence, hydrogen or methyl;
z is an integer from 10 to 75;
X is —H, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH; and
M⁺ is a cation.

In some embodiments, z can be 15 to 50. In some embodiments, R¹ is a linear or branched C₁-C₆ alkyl group, such as a linear or branched C₁-C₅ alkyl group, or a linear or branched C₁-C₄ alkyl group. In some embodiments, R¹ is a C₁-C₃ alkyl group. In some embodiments, R¹ is methyl. In some embodiments, R¹ is ethyl. In some embodiments, R¹ is phenyl. In some embodiments, when R¹ is a phenyl group, the phenyl group can be substituted with one C₁-C₂ alkyl group. In some embodiments, when R¹ is a phenyl group, the phenyl group can be substituted with two C₁-C₂ alkyl groups. In some embodiments, X can be —SO₃M⁺, —SO₃H, —CH₂C(O)O⁻M⁺, or —CH₂C(O)OH; and M⁺ is a cation.

In some embodiments, wherein the compound is defined by Formula II

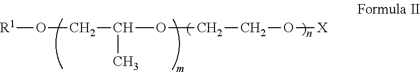

wherein
R¹ is a C₁-C₈ alkyl group or C₆ aryl group;
m is an integer from 1 to 50 and n is an integer from 1 to 50, with the proviso that m+n is from 10 to 75;
X is —H, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH; and
M⁺ is a cation.

In some embodiments, m can be from 5 to 25, such as from 5 to 20 or from 10 to 20. In some embodiments, n can be from 5 to 35, such as from 5 to 30 or from 10 to 25. In some embodiments, the sum of m and n (m+n) can be from 15 to 50.

Described herein are also compositions including a compound described herein and water. In some embodiments, the compound can be present in the composition in an amount of from 0.05% to 2% by weight, based on the total weight of the composition. In some embodiments, the composition can further include an additional surfactant. In some embodiments, the additional surfactant comprises an anionic surfactant, a non-ionic surfactant, a cationic surfactant, a zwitteronic surfactant, or any combination thereof. In some embodiments, the additional surfactant is present in the composition in an amount of from 0.05% to 2% by weight, based on the total weight of the composition. In some embodiments, the composition can further include a viscosity-enhancing water-soluble polymer. In some embodiments, the composition can further include an alkali agent. In some embodiments, the composition has a pH of from 10 to 12. In some embodiments, the composition can further include a co-solvent. In some embodiments, the composition can have a salinity of at least 5,000 ppm.

Described herein are also methods of displacing an unrefined petroleum material in contact with a solid material, said method comprising:
contacting the unrefined petroleum material with the compound described herein or a composition described herein, wherein the unrefined petroleum material is in contact with the solid material; and
allowing the unrefined petroleum material to separate from the solid material, thereby displacing the unrefined petroleum material in contact with the solid material.

Described herein are also methods of displacing an unrefined petroleum material in contact with a solid material, said method comprising:
(i) contacting the unrefined petroleum material with the compound described herein or the composition described herein, wherein the unrefined petroleum material is in contact with the solid material; and
(ii) allowing the unrefined petroleum material to separate from the solid material, thereby displacing the unrefined petroleum material in contact with the solid material.

Described herein are also methods for recovering hydrocarbons from a subterranean formation, the method comprising:
(a) introducing the compound described herein or the composition described herein through a wellbore into the subterranean formation; and
(b) producing fluids from the subterranean formation; wherein the hydrocarbons in the subterranean formation comprise live oil.

In some embodiments, step (a) comprises injecting the compound described herein or the composition described herein through an injection wellbore into the subterranean formation. In some embodiments, step (b) comprises producing the fluids from a production wellbore spaced apart from the injection wellbore a predetermined distance and in fluid communication with the subterranean formation; wherein injection of the compound described herein or the composition described herein increases a flow of hydrocarbons to the production wellbore.

In some embodiments, the unrefined petroleum includes live oil. In some embodiments, the live oil has a gas/oil ratio (GOR) of from 100 to 10,000 scf/bbl.

The surfactants and methods of using thereof described herein are further described in the attached Appendix, which is hereby incorporated by reference in its entirety.

### DESCRIPTION OF DRAWINGS

FIG. 1 shows a general representation of surfactant structure.

FIG. 2 shows an oil scan using 0.3% surfactant blend of 2-ethylhexanol(2EH)-40PO-60EO-CH2COONa with alkyl benzene sulfonate as co-surfactant FIG. 3 shows an oil scan using 0.3% surfactant blend of 2-ethylhexanol(2EO)-40PO-40EO-H with alkyl benzene sulfonate as co-surfactant.

FIG. 4 shows a plot of solubilization ratios for dead oil using a conventional formulation that includes (1 wt % surfactant) 0.5 wt % C₁₈-35PO-20EO carboxylate with 0.5 wt % ABS (co-surfactant) and 0.5 wt % phenol-2EO (co-solvent) at 74° C. and 1 atm.

FIG. 5 shows an image of the phase behavior for live oil using a conventional formulation that includes (1 wt % surfactant) 0.5 wt % C₁₈-35PO-20EO carboxylate with 0.5 wt % ABS (co-surfactant) and 0.5 wt % phenol-2EO (co-solvent) at 74° C. and 5700 psi at (7,489 ppm TDS, 10,472 ppm TDS, 13,455 ppm TDS, and 15,419 ppm TDS).

FIG. 6 shows an image of the phase behavior for live oil using a formulation #1 including (1 wt % surfactant) 0.5 wt % C₃-21PO-10EO sulfate with 0.5 wt % light IOS (co-surfactant) at 74° C. and 5700 psi at (13,455 ppm TDS and 15,419 ppm TDS).

FIG. 7 shows an image of the phase behavior for live oil using a formulation #2 including C₃O-21PO-10EO sulfate with C₆-diphenyl disulfonate (co-surfactant) with 8 different ratios of surfactant to co-surfactant at 74° C. and 5700 psi.

FIG. 8A-8C shows images of the aqueous stability (FIG. 8A), and phase behavior for dead oil (FIG. 8B) and live oil (FIG. 8C) using a formulation #4 including C₃O-21PO-45EO-OH with glycerin 6-PO (co-solvent) with 6 different ratios of surfactant to co-solvent at 74° C. and 5700 psi.

FIG. 9A-9C shows images of the aqueous stability (FIG. 9A), and phase behavior for dead oil (FIG. 9B) and live oil (FIG. 9C) using a formulation #4 including C₃O-21PO-45EO-OH with glycerin 6-PO (co-solvent) in high-salinity produced waters at 74° C. and 5700 psi. Six different ratios of surfactant to co-solvent where tested and the surfactant concentration was diluted to 50% compared to surfactant concentration tested in FIG. 8.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) can includes 10% and also includes 20%, and includes percentages in between 10% and 20%, unless explicitly stated otherwise herein.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “contacting” as used herein, refers to materials or compounds being sufficiently close in proximity to react or interact. For example, in methods of contacting an unrefined petroleum material, a hydrocarbon material bearing formation, and/or a well bore, the term “contacting” can include placing a compound (e.g., a surfactant) or an aqueous composition (e.g., chemical, surfactant or polymer) within a hydrocarbon material-bearing formation using any suitable manner known in the art (e.g., pumping, injecting, pouring, releasing, displacing, spotting or circulating the chemical into a well, well bore or hydrocarbon bearing formation).

The terms “unrefined petroleum” and “crude oil” are used interchangeably and in keeping with the plain ordinary usage of those terms. “Unrefined petroleum” and “crude oil” may be found in a variety of petroleum reservoirs (also referred to herein as a “reservoir,” “oil field deposit” “deposit” and the like) and in a variety of forms including oleaginous materials, oil shales (i.e., organic-rich fine-grained sedimentary rock), tar sands, light oil deposits, heavy oil deposits, and the like. “Crude oils” or “unrefined petroleums” generally refer to a mixture of naturally occurring hydrocarbons that may be refined into diesel, gasoline, heating oil, jet fuel, kerosene, and other products called fuels or petrochemicals. Crude oils or unrefined petroleums are named according to their contents and origins, and are classified according to their per unit weight (specific gravity). Heavier crudes generally yield more heat upon burning, but have lower gravity as defined by the American Petroleum Institute (API) (i.e., API gravity) and market price in comparison to light (or sweet) crude oils. Crude oil may also be characterized by its Equivalent Alkane Carbon Number (EACN). The term “API gravity” refers to the measure of how heavy or light a petroleum liquid is compared to water. If an oil's API gravity is greater than 10, it is lighter and floats on water, whereas if it is less than 10, it is heavier and sinks. API gravity is thus an inverse measure of the relative density of a petroleum liquid and the density of water. API gravity may also be used to compare the relative densities of petroleum liquids. For example, if one petroleum liquid floats on another and is therefore less dense, it has a greater API gravity.

Crude oils vary widely in appearance and viscosity from field to field. They range in color, odor, and in the properties they contain. While all crude oils are mostly hydrocarbons, the differences in properties, especially the variation in molecular structure, determine whether a crude oil is more or less easy to produce, pipeline, and refine. The variations may even influence its suitability for certain products and the quality of those products. Crude oils are roughly classified into three groups, according to the nature of the hydrocarbons they contain. (i) Paraffin-based crude oils contain higher molecular weight paraffins, which are solid at room temperature, but little or no asphaltic (bituminous) matter. They can produce high-grade lubricating oils. (ii) Asphaltene based crude oils contain large proportions of asphaltic matter, and little or no paraffin. Some are predominantly naphthenes and so yield lubricating oils that are sensitive to temperature changes than the paraffin-based crudes. (iii) Mixed based crude oils contain both paraffin and naphthenes, as well as aromatic hydrocarbons. Most crude oils fit this latter category.

“Reactive” crude oil, as referred to herein, is crude oil containing natural organic acidic components (also referred to herein as unrefined petroleum acid) or their precursors such as esters or lactones. These reactive crude oils can generate soaps (carboxylates) when reacted with alkali. More terms used interchangeably for crude oil throughout this disclosure are hydrocarbon material or active petroleum material. An “oil bank” or “oil cut” as referred to herein, is the crude oil that does not contain the injected chemicals and is pushed by the injected fluid during an enhanced oil recovery process. A “nonactive oil,” as used herein, refers to an oil that is not substantially reactive or crude oil not containing significant amounts of natural organic acidic components or their precursors such as esters or lactones such that significant amounts of soaps are generated when reacted with alkali. A nonactive oil as referred to herein includes oils having an acid number of less than 0.5 mg KOH/g of oil.

“Unrefined petroleum acids” as referred to herein are carboxylic acids contained in active petroleum material (reactive crude oil). The unrefined petroleum acids contain C₁-C₂₀ alkyl chains, including napthenic acid mixtures. The recovery of such “reactive” oils may be performed using alkali (e.g., NaOH or Na₂CO₃) in a surfactant composition. The alkali reacts with the acid in the reactive oil to form soap in situ. These in situ generated soaps serve as a source of surfactants minimizing the levels of added surfactants, thus enabling efficient oil recovery from the reservoir.

The term “polymer” refers to a molecule having a structure that essentially includes the multiple repetitions of units derived, actually or conceptually, from molecules of low relative molecular mass. In some embodiments, the polymer is an oligomer.

The term “productivity” as applied to a petroleum or oil well refers to the capacity of a well to produce hydrocarbons (e.g., unrefined petroleum); that is, the ratio of the hydrocarbon flow rate to the pressure drop, where the pressure drop is the difference between the average reservoir pressure and the flowing bottom hole well pressure (i.e., flow per unit of driving force).

The term “oil solubilization ratio” is defined as the volume of oil solubilized divided by the volume of surfactant in microemulsion. All the surfactant is presumed to be in the microemulsion phase. The oil solubilization ratio is applied for Winsor type I and type III behavior. The volume of oil solubilized is found by reading the change between initial aqueous level and excess oil (top) interface level. The oil solubilization ratio is calculated as follows:

$${σ}_{o} = \frac{{V}_{o}}{{V}_{s}}$$

where σₒ is the oil solubilization ratio, Vₒ is the volume of oil solubilized, and Vₛ is the volume of surfactant.

The term “water solubilization ratio” is defined as the volume of water solubilized divided by the volume of surfactant in microemulsion. All the surfactant is presumed to be in the microemulsion phase. The water solubilization ratio is applied for Winsor type III and type II behavior. The volume of water solubilized is found by reading the change between initial aqueous level and excess water (bottom) interface level. The water solubilization parameter is calculated as follows:

$${σ}_{w} = \frac{{V}_{w}}{{V}_{s}}$$

where σ_{w} is the water solubilization ratio, V_{w} is the volume of oil solubilized, and Vₛ is the volume of surfactant.

The optimum solubilization ratio occurs where the oil and water solubilization ratios are equal. The coarse nature of phase behavior screening often does not include a data point at optimum, so the solubilization ratio curves are drawn for the oil and water solubilization ratio data and the intersection of these two curves is defined as the optimum. The following is true for the optimum solubilization ratio:
σₒ-σ_{w}-σ*
   where σ* is the optimum solubilization ratio.

The term “solubility” or “solubilization” in general refers to the property of a solute, which can be a solid, liquid or gas, to dissolve in a solid, liquid or gaseous solvent thereby forming a homogenous solution of the solute in the solvent. Solubility occurs under dynamic equilibrium, which means that solubility results from the simultaneous and opposing processes of dissolution and phase joining (e.g., precipitation of solids). The solubility equilibrium occurs when the two processes proceed at a constant rate. The solubility of a given solute in a given solvent typically depends on temperature. For many solids dissolved in liquid water, the solubility increases with temperature. In liquid water at high temperatures, the solubility of ionic solutes tends to decrease due to the change of properties and structure of liquid water. In more particular, solubility and solubilization as referred to herein is the property of oil to dissolve in water and vice versa.

“Viscosity” refers to a fluid's internal resistance to flow or being deformed by shear or tensile stress. In other words, viscosity may be defined as thickness or internal friction of a liquid. Thus, water is “thin”, having a lower viscosity, while oil is “thick”, having a higher viscosity. More generally, the less viscous a fluid is, the greater its ease of fluidity.

The term “salinity” as used herein, refers to concentration of salt dissolved in an aqueous phases. Examples for such salts are without limitation, sodium chloride, magnesium and calcium sulfates, and bicarbonates. In more particular, the term salinity as it pertains to the present invention refers to the concentration of salts in brine and surfactant solutions.

The term “aqueous solution or aqueous formulation” refers to a solution in which the solvent is water. The term “emulsion, emulsion solution or emulsion formulation” refers to a mixture of two or more liquids which are normally immiscible. A non-limiting example for an emulsion is a mixture of oil and water.

The term “co-solvent,” as used herein, refers to a compound having the ability to increase the solubility of a solute (e.g., a surfactant as disclosed herein) in the presence of an unrefined petroleum acid. In some embodiments, the co-solvents provided herein have a hydrophobic portion (alkyl or aryl chain), a hydrophilic portion (e.g., an alcohol) and an alkoxy portion.

The term “interfacial tension” or “IFT” as used herein refers to the surface tension between test oil and water of different salinities containing a surfactant formulation at different concentrations. Typically, interfacial tensions are measured using a spinning drop tensiometer or calculated from phase behavior experiments.

The term “contacting” as used herein, refers to materials or compounds being sufficiently close in proximity to react or interact. For example, in methods of contacting an unrefined petroleum material, a hydrocarbon-bearing formation, and/or a wellbore, the term “contacting” can include placing a compound (e.g., a surfactant) or an aqueous composition (e.g., chemical, surfactant or polymer) within a hydrocarbon-bearing formation using any suitable manner known in the art (e.g., pumping, injecting, pouring, releasing, displacing, spotting or circulating the chemical into a well, wellbore or hydrocarbon-bearing formation).

The term “live oil,” as used herein, refers generally to an oil containing dissolved gas (e.g., methane) in solution.

### Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. Ph in Formula I refers to a phenyl group.

The prefix Cₙ-Cₘ preceding a group or moiety indicates, in each case, the possible number of carbon atoms in the group or moiety that follows.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, heteroatoms present in a compound or moiety, such as nitrogen, can have hydrogen substituents and/or any permissible substituents of organic compounds described herein which satisfy the valency of the heteroatom. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound (e.g., a compound that does not spontaneously undergo transformation such as by rearrangement, cyclization, elimination, etc.

The term “optionally substituted,” as used herein, means that substitution with an additional group is optional and therefore it is possible for the designated atom to be unsubstituted. Thus, by use of the term “optionally substituted” the disclosure includes examples where the group is substituted and examples where it is not.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, and 1-ethyl-2-methyl-propyl. Alkyl substituents may be unsubstituted or substituted with one or more chemical moieties. The alkyl group can be substituted with one or more groups including, but not limited to, hydroxy, halogen, acyl, alkyl, alkoxy, alkenyl, alkynyl, aryl, heteroaryl, acyl, aldehyde, amino, carboxylic acid, ester, ether, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied. The alkyl group can also include one or more heteroatoms (e.g., from one to three heteroatoms) incorporated within the hydrocarbon moiety. Examples of heteroatoms include, but are not limited to, nitrogen, oxygen, sulfur, and phosphorus.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. The term “alkylthiol” specifically refers to an alkyl group that is substituted with one or more thiol groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below.

As used herein, the term “aryl,” as well as derivative terms such as aryloxy, refers to groups that include a monovalent aromatic carbocyclic group of from 3 to 20 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphthyl, phenylcyclopropyl, and indanyl. In some embodiments, the aryl group can be a phenyl, indanyl or naphthyl group. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, cycloalkyl, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “acyl” as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

As used herein, the term “alkoxy” refers to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

As used herein, Me refers to a methyl group; OMe refers to a methoxy group; and i-Pr refers to an isopropyl group.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

The term “substituted” refers to a molecule wherein at least one hydrogen atom is replaced with a substituent. When substituted, one or more of the groups are “substituents.” The molecule can be multiply substituted. In the case of an oxo substituent (“═O”), two hydrogen atoms are replaced. Example substituents within this context can include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO₂Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context can be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

Reference will now be made in detail to specific aspects of the disclosed materials, compounds, compositions, articles, and methods, examples of which are illustrated in the accompanying Examples and Figures.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

### Compounds

Provided herein are compounds defined by Formula I below

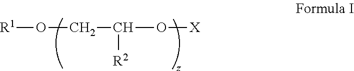

wherein
R¹ is a C₁-C₈ alkyl group or C₆ aryl group optionally substituted with one or two C₁-C₂ alkyl groups;
R² is, independently for each occurrence, hydrogen or methyl;
z is an integer from 10 to 75;
X is —H, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH; and
M⁺ is a cation.

In some embodiments, z can be an integer from 10 to 75 (e.g. from 10 to 70, from 15 to 75, from 20 to 75, from 25 to 75, from 30 to 75, from 30 to 75, from 40 to 75, from 50 to 75, from 60 to 75, from 70 to 75, from 10 to 70, from 15 to 70, from 20 to 70, from 25 to 70, from 30 to 70, from 40 to 70, from 50 to 70, from 10 to 50, from 15 to 50, from 20 to 50, from 25 to 50, from 30 to 50, from 40 to 50, from 10 to 40, from 15 to 40, from 20 to 40, from 25 to 40, from 30 to 40, from 10 to 35, from 15 to 35, from 20 to 35, from 25 to 35, from 10 to 30, from 15 to 30, from 20 to 30, from 25 to 30, from 10 to 25, from 15 to 25, from 20 to 25, from 10 to 20, from 15 to 20, from 10 to 15). In some embodiments, z can be 10, 15, 20, 25, 30, 35, 40, 45, 50, 55, 60, 65, 70, or 75. In some embodiments, z can be 15 to 50.

In some embodiments, the compound is defined by Formula II

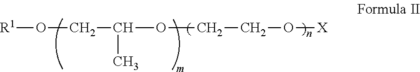

wherein
R¹ is a C₁-C₈ alkyl group or C₆ aryl group;
m is an integer from 1 to 50 and n is an integer from 1 to 50, with the proviso that m+n is from 10 to 75;
X is —H, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH; and
M⁺ is a cation.

In some embodiments, R¹ is a linear or branched C₁-C₆ alkyl group, such as a linear or branched C₁-C₅ alkyl group, or a linear or branched C₁-C₄ alkyl group. In some embodiments, R¹ is a C₁-C₃ alkyl group. In some embodiments, R¹ is methyl. In some embodiments, R¹ is ethyl. In some embodiments, R¹ is phenyl. In some embodiments, when R¹ is a phenyl group, the phenyl group can be substituted with one C₁-C₂ alkyl group. In some embodiments, when R¹ is a phenyl group, the phenyl group can be substituted with two C₁-C₂ alkyl groups.

In some embodiments, the compound is defined by Formula III

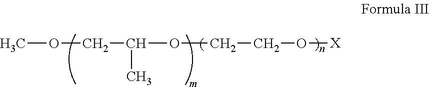

wherein
m is an integer from 1 to 50 and n is an integer from 1 to 50, with the proviso that m+n is from 10 to 75;
X is —H, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH; and
M⁺ is a cation.

In some embodiments, the compound is defined by Formula IV

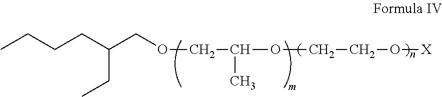

wherein
m is an integer from 1 to 50 and n is an integer from 1 to 50, with the proviso that m+n is from 10 to 75;
X is —H, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH; and
M⁺ is a cation.

In some embodiments, the compound is defined by Formula IV

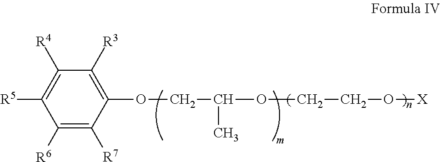

wherein
R³-R⁷ are each independently hydrogen or a C₁-C₂ alkyl;
m is an integer from 1 to 50 and n is an integer from 1 to 50, with the proviso that m+n is from 10 to 75;
X is —H, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH; and
M⁺ is a cation. In some embodiments, wherein at least one R³-R⁷ is a C₁-C₂ alkyl group. In some embodiments, wherein at least two of R³-R⁷ are a C₁-C₂ alkyl group.

In some embodiments, X can be —SO₃M⁺, —SO₃H, —CH₂C(O)O⁻M⁺, or —CH₂C(O)OH; and M⁺ is a cation. In some embodiments, X can be —SO₃M⁺; and M⁺ is a cation. In some embodiments, X can be —CH₂C(O)O⁻M⁺; and M⁺ is a cation. In some embodiments, X can be —SO₃H. In some embodiments, X can be-CH₂C(O)OH.

In some embodiments, X can be CH₂CH(OH)CH₂—SO₃M⁺ or CH₂CH(OH)CH₂—SO₃H, and M⁺ is a cation.

In some embodiments, X can be —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH, and M⁺ is a cation.

In some embodiments, m can be an integer from 1 to 50, (e.g., from 10 to 20, from 10 to 25, from 10 to 30, from 10 to 35, from 10 to 40, from 10 to 45, from 10 to 50, from 15 to 25, from 15 to 30, from 15 to 35, from 15 to 40, from 15 to 50, from 20 to 30, from 20 to 35, from 20 to 40, from 20 to 50, from 25 to 30, from 25 to 40, from 25 to 50, from 30 to 40, from 30 to 45, from 30 to 50, from 40 to 50, from 5 to 20, from 5 to 25, from 5 to 30, from 5 to 40, or from 5 to 50). In some embodiments, m can be 21.

In some embodiments, n can be an integer from 1 to 50, (e.g., from 10 to 20, from 10 to 25, from 10 to 30, from 10 to 35, from 10 to 40, from 10 to 45, from 10 to 50, from 15 to 25, from 15 to 30, from 15 to 35, from 15 to 40, from 15 to 50, from 20 to 30, from 20 to 35, from 20 to 40, from 20 to 50, from 25 to 30, from 25 to 40, from 25 to 50, from 30 to 40, from 30 to 45, from 30 to 50, from 40 to 50, from 5 to 20, from 5 to 25, from 5 to 30, from 5 to 35, from 5 to 40, or from 5 to 50). In some embodiments, n can be 20. In some embodiments, n can be 45.

In some embodiments, the sum of m and n (m+n) is from 10 to 75 (e.g., from 10 to 20, from 10 to 25, from 10 to 30, from 10 to 35, from 10 to 40, from 10 to 45, from 10 to 50, from 10 to 60, from 10 to 70, from 15 to 25, from 15 to 30, from 15 to 35, from 15 to 40, from 15 to 50, from 15 to 60, from 15 to 75, from 20 to 30, from 20 to 35, from 20 to 40, from 20 to 50, from 20 to 60, from 20 to 75, from 25 to 30, from 25 to 40, from 25 to 50, from 25 to 60, from 25 to 75, from 30 to 40, from 30 to 45, from 30 to 50, from 30 to 60, from 30 to 75, from 40 to 50, from 30 to 60, from 30 to 75.

### Composition

As described above, the compounds described herein can be used in EOR formulations to provide aqueous stability and ultra-low interfacial tension region.

Accordingly, also provided are aqueous compositions for use in EOR that comprise the compounds described herein. For example, provided herein are aqueous composition that comprise a compound described herein (e.g., a compound of Formula I) and water. Additional components, including viscosity-enhancing water-soluble polymers, alkali agents, additional surfactants, co-solvents, and combinations thereof, can be present in the aqueous compositions.

In some embodiments, the aqueous composition can further comprise a surfactant. A surfactant, as used herein, is a compound within the aqueous composition that functions as a surface active agent when the aqueous composition is in contact with a crude oil (e.g., an unrefined petroleum). The surfactant can act to lower the interfacial tension and/or surface tension of the unrefined petroleum. In some embodiments, the surfactant and the compound of Formula I are present in synergistic surface active amounts. A “synergistic surface active amount,” as used herein, means that a compound of Formula I and the surfactant are present in amounts in which the oil surface activity (interfacial tension lowering effect and/or surface tension lowering effect on crude oil when the aqueous composition is added to the crude oil) of the compound and surfactant combined is greater than the additive oil surface activity of the surfactant individually and the compound individually. In some cases, the oil surface activity of the compound and surfactant combination is 10%, 20%, 30%, 40%, 50%, 60%, 70%, 80%, 90% or 100% more than the additive oil surface activity of the surfactant individually and the compound individually. In some embodiments, the oil surface activity of the compound and surfactant combination is 2, 3, 4, 5, 6, 7, 8, 9 or 10 times more than the additive oil surface activity of the surfactant individually and the compound individually.

In another embodiment, the compound and surfactant are present in a surfactant stabilizing amount. A “surfactant stabilizing amount” means that the compound and the surfactant are present in an amount in which the surfactant degrades at a slower rate in the presence of the compound than in the absence of the compound, and/or the compound degrades at a slower rate in the presence of the surfactant than in the absence of the surfactant. The rate of degradation may be 10%, 20%, 30%, 40%, 50%, 60%, 70%, 80%, 90% or 100% slower. In some embodiments, the rate of degradation is 2, 3, 4, 5, 6, 7, 8, 9 or 10 times slower.

In another embodiment, the compound and surfactant are present in a synergistic solubilizing amount. A “synergistic solubilizing amount” means that the compound and the surfactant are present in an amount in which the compound is more soluble in the presence of the surfactant than in the absence of the surfactant, and/or the surfactant is more soluble in the presence of the compound than in the absence of the compound. The solubilization may be 10%, 20%, 30%, 40%, 50%, 60%, 70%, 80%, 90% or 100% higher. In some embodiment, the solubilization is 2, 3, 4, 5, 6, 7, 8, 9 or 10 times higher. In some embodiments, the compound is present in an amount sufficient to increase the solubility of the surfactant in the aqueous composition relative to the absence of the compound. In other words, in the presence of a sufficient amount of the compound, the solubility of the surfactant in the aqueous composition is higher than in the absence of the compound. In other embodiments, the surfactant is present in an amount sufficient to increase the solubility of the compound in the aqueous composition relative to the absence of the surfactant. Thus, in the presence of a sufficient amount of the surfactant the solubility of the compound in the aqueous solution is higher than in the absence of the surfactant.

In some embodiments, a single type of surfactant is in the aqueous composition. In other embodiments, a surfactant can comprise a blend of surfactants (e.g., a combination of two or more surfactants). The surfactant blend can comprise a mixture of a plurality of surfactant types. For example, the surfactant blend can include at least two surfactant types, at least three surfactant types, at least four surfactant types, at least five surfactant types, at least six surfactant types, or more. In some embodiments, the surfactant blend can include from two to six surfactant types (e.g., from two to five surfactant types, from two to four surfactant types, from two to three surfactant types, from three to six surfactant types, or from three to five surfactant types). The surfactant types can be independently different (e.g., anionic or cationic surfactants; two anionic surfactants having a different hydrocarbon chain length but are otherwise the same; a sulfate and a sulfonate surfactant that that the same hydrocarbon chain length and are otherwise the same, etc.). Therefore, a person having ordinary skill in the art will immediately recognize that the terms “surfactant” and “surfactant type(s)” have the same meaning and can be used interchangeably.

In some embodiments, the surfactant can comprise an anionic surfactant, a non-ionic surfactant, a zwitterionic surfactant, a cationic surfactant, or a combination thereof. In some embodiments, the surfactant can comprise an anionic surfactant, a non-ionic surfactant, or a combination thereof. In some embodiments, the surfactant can comprise a plurality of anionic surfactants. In some embodiments, the surfactant can comprise a zwitterionic surfactant. “Zwitterionic” or “zwitterion” as used herein refers to a neutral molecule with a positive (or cationic) and a negative (or anionic) electrical charge at different locations within the same molecule. Examples of zwitterionic surfactants include without limitation betains and sultains.

The surfactant can be any appropriate surfactant useful in the field of enhanced oil recovery. For example, in some embodiments, the surfactant can comprise an internal olefin sulfonate (IOS), an alpha olefin sulfonate (AOS), an alkyl aryl sulfonate (ARS), an alkane sulfonate, a petroleum sulfonate, an alkyl diphenyl oxide (di)sulfonate, an alcohol sulfate, an alkoxy sulfate, an alkoxy sulfonate, an alcohol phosphate, an alkoxy phosphate, a sulfosuccinate ester, an alcohol ethoxylate, an alkyl phenol ethoxylate, a quaternary ammonium salt, a betaine or sultaine. The surfactant as provided herein, can also be a soap.

In embodiments, the surfactant can comprise an anionic surfactant. For example, the surfactant can comprise an anionic surfactant selected from the group consisting of alkoxy carboxylate surfactants, alkoxy sulfate surfactants, alkoxy sulfonate surfactants, alkyl sulfonate surfactants, aryl sulfonate surfactants, olefin sulfonate surfactants, and combinations thereof. In embodiments, the anionic surfactant can comprise an anionic surfactant blend. Where the anionic surfactant is an anionic surfactant blend, the aqueous composition includes a plurality (i.e., more than one) type of anionic surfactant.

In some embodiments, the surfactant can be a polyol alkoxylate surfactant. The polyol alkoxylate surfactant can be defined by Formula A

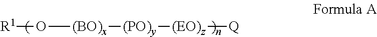

wherein
BO represents —CH₂—CH(ethyl)-O— or —CH₃CH(O—)CH₃;
PO represents —CH₂—CH(methyl)-O—;
EO represents —CH₂—CH₂—O—;
R¹ represents a C₁-C₈ alkyl group (e.g., a C₁-C₆ alkyl group) or a cyclic structure derived from alkoxylation of an alkyl monoglucoside, an alkyl polyglucoside, a monosaccharide, a disaccharide, or a polysaccharide;
n is an integer from 2 to 6;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
x is an integer from 0 to 15;
y is an integer from 11 to 60; and
z is an integer from 0 to 100.

In some embodiments, n can be an integer from 3 to 6, from 3 to 5, from 3 to 4, from 4 to 5, or from 4 to 6. In some embodiments, n can be an integer from 3 to 6. In some embodiments, n can be 3, 4, 5, or 6. In some embodiments, n is 3.

In some embodiments, R¹ can be a C₁-C₆ alkyl, C₁-C₅ alkyl, C₁-C₄ alkyl, C₁-C₃ alkyl, C₁-C₂ alkyl, C₂-C₆ alkyl, C₂-C₅ alkyl, C₂-C₄ alkyl, C₂-C₃ alkyl, C₃-C₆ alkyl, C₃-C₅ alkyl, C₃-C₄ alkyl, C₄-C₅ alkyl, C₄-C₆ alkyl, or C₅-C₆ alkyl group. In some embodiments, R¹ can be a C₃-C₆ alkyl group.

In some embodiments, the compound of Formula A can be defined by Formula B below

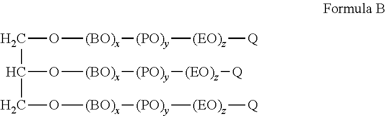

wherein
BO represents —CH₂—CH(ethyl)-O— or —CH₃CH(O—)CH₃;
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O-M⁺, or —CH₂C(O)OH;
M⁺, when present, is a cation;
x is an integer from 0 to 15;
y is an integer from 11 to 60; and
z is an integer from 0 to 100.

In some embodiments, the compound of Formula A can be defined by Formula C below

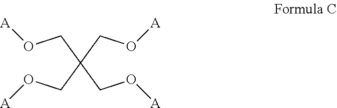

wherein
A represents -(BO)ₓ-(PO)_{y}-(EO)_{z}-Q
BO represents —CH₂—CH(ethyl)-O— or —CH₃CH(O—)CH₃;
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
x is an integer from 0 to 15;
y is an integer from 11 to 60; and
z is an integer from 0 to 100.

In some embodiments, the compound of Formula A can be defined by Formula C-a below

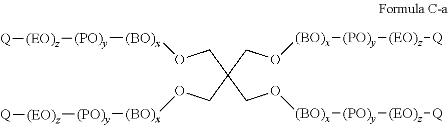

wherein
BO represents —CH₂—CH(ethyl)-O— or —CH₃CH(O—)CH₃;
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
x is an integer from 0 to 15;
y is an integer from 11 to 60; and
z is an integer from 0 to 100.

In some embodiments, the compound of Formula A can be defined by Formula D below

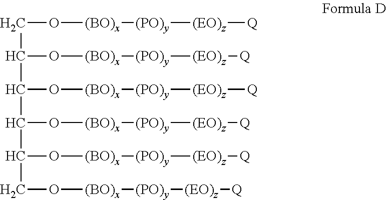

wherein
BO represents —CH₂—CH(ethyl)-O— or —CH₃CH(O—)CH₃;
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
x is an integer from 0 to 15;
y is an integer from 11 to 60; and
z is an integer from 0 to 100.

In some embodiments, the compound of Formula A can be defined by Formula E below

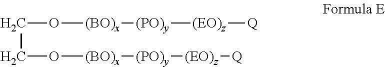

wherein
BO represents —CH₂—CH(ethyl)-O— or —CH₃CH(O—)CH₃;
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
x is an integer from 0 to 15;
y is an integer from 11 to 60; and
z is an integer from 0 to 100.

In some embodiments, the compound of Formula A can be defined of Formula F below

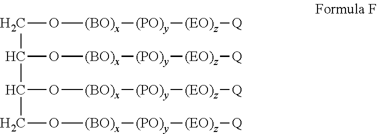

wherein
BO represents —CH₂—CH(ethyl)-O— or —CH₃CH(O—)CH₃;
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
x is an integer from 0 to 15;
y is an integer from 11 to 60; and
z is an integer from 0 to 100.

In some embodiments, the compound of Formula A can be defined of Formula G below

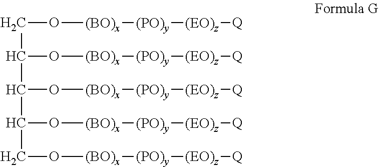

wherein
BO represents —CH₂—CH(ethyl)-O— or —CH₃CH(O—)CH₃;
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
x is an integer from 0 to 15;
y is an integer from 11 to 60; and
z is an integer from 0 to 100.

In some embodiments, the compound of Formula A can be defined by Formula H below

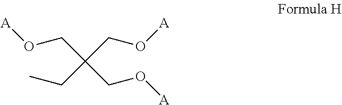

wherein
A represents -(BO)ₓ-(PO)_{y}-(EO)_{z}-Q
BO represents —CH₂—CH(ethyl)-O— or —CH₃CH(O—)CH₃;
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
x is an integer from 0 to 15;
y is an integer from 11 to 60; and
z is an integer from 0 to 100.

In some embodiments, the compound of Formula A can be defined by Formula D-a below

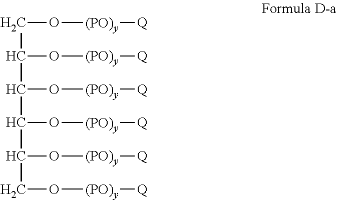

wherein
PO represents —CH₂—CH(methyl)-O—;
Q is —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation; and
y is an integer from 11 to 60.

In some embodiments, the compound of Formula A can be defined by Formula E-a below

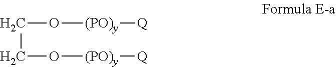

wherein
PO represents —CH₂—CH(methyl)-O—;
Q is —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation; and
y is an integer from 11 to 60.

In some embodiments, the compound of Formula A can be defined by Formula B-a below

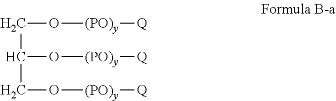

wherein
PO represents —CH₂—CH(methyl)-O—;
Q is —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation; and
y is an integer from 11 to 60.

In some embodiments, the compound of Formula A can be defined by Formula F-a below

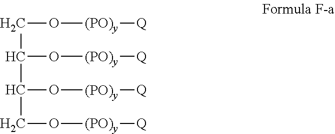

wherein
PO represents —CH₂—CH(methyl)-O—;
Q is —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation; and
y is an integer from 11 to 60.

In some embodiments, the compound of Formula A can be defined by Formula G-a below

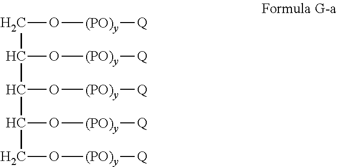

wherein
PO represents —CH₂—CH(methyl)-O—;
Q is —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation; and
y is an integer from 11 to 60.

In some embodiments, the compound of Formula A can be defined by Formula H-a below

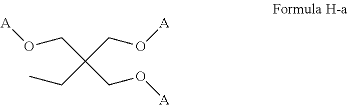

wherein
A represents -(PO)_{y}-Q
PO represents —CH₂—CH(methyl)-O—;
R¹ represents a C₁-C₆ alkyl group;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60.

In some embodiments, the compound of Formula A can be defined by Formula B-b below

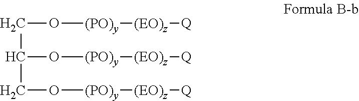

wherein
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, the compound of Formula A can be defined by Formula C-b below

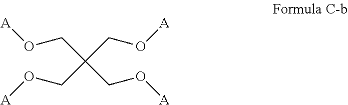

wherein
A represents -(PO)_{y}-(EO)_{z}-Q
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, the compound of Formula A can be defined by Formula C-c below

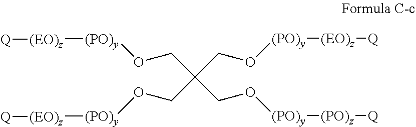

wherein
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, the compound of Formula A can be defined by Formula D-b below

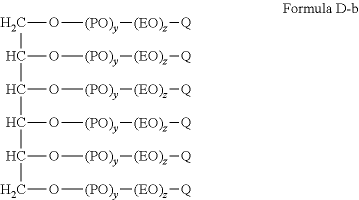

wherein
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, the compound of Formula A can be defined by Formula E-b below

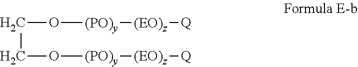

wherein
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, the compound of Formula A can be defined of Formula F-b below

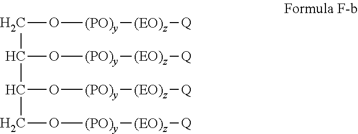

wherein
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, the compound of Formula A can be defined of Formula G-b below

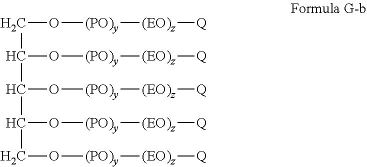

wherein
   PO represents —CH₂—CH(methyl)-O—;
   EO represents —CH₂—CH₂—O—;
   Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
   M⁺, when present, is a cation;
y is an integer from 11 to 60; and
   z is an integer from 0 to 100.

In some embodiments, the compound of Formula A can be defined by Formula H-b below

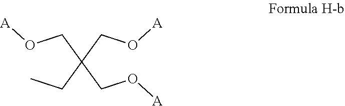

wherein
A represents -(PO)_{y}-(EO)_{z}-Q
PO represents —CH₂—CH(methyl)-O—;
EO represents —CH₂—CH₂—O—;
R¹ represents a C₁-C₆ alkyl group;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, the compound of Formula A can be defined by Formula B-c below

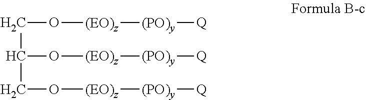

wherein
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, the compound of Formula I can be defined by Formula C-d below

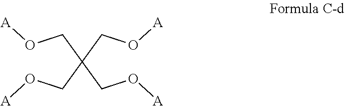

wherein
A represents -(EO)_{z}-(PO)_{y}-Q
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, the compound of Formula A can be defined by Formula C-e below

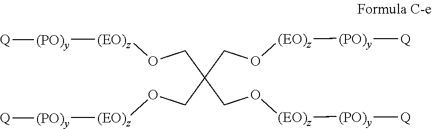

wherein
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, the compound of Formula A can be defined by Formula D-c below

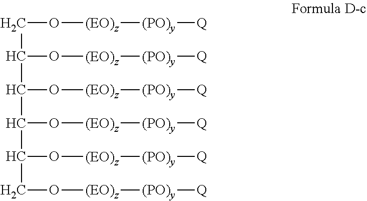

wherein
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, the compound of Formula A can be defined by Formula E-c below

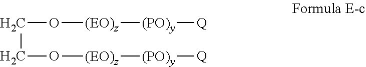

wherein
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, the compound of Formula A can be defined of Formula F-c below

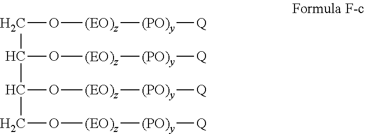

wherein
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, the compound of Formula A can be defined of Formula G-c below

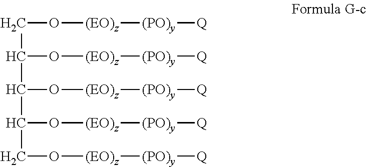

wherein
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, the compound of Formula A can be defined by Formula H-c below

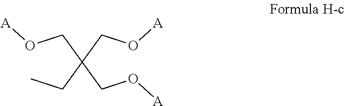

wherein
A represents -(EO)_{z}-(PO)_{y}-Q
PO represents —CH₂—CH(methyl)-O—;
EO represents —CH₂—CH₂—O—;
R¹ represents a C₁-C₆ alkyl group;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
y is an integer from 11 to 60; and
z is an integer from 0 to 100.

In some embodiments, Q can be —SO₃M⁺, —SO₃H, —CH₂C(O)O⁻M⁺, or —CH₂C(O)OH; and M⁺ is a cation. In some embodiments, Q can be —SO₃M⁺; and M⁺ is a cation. In some embodiments, Q can be-CH₂C(O)O⁻M⁺; and M⁺ is a cation. In some embodiments, Q can be —SO₃H. In some embodiments, Q can be-CH₂C(O)OH. In some embodiments, Q can be CH₂CH(OH)CH₂—SO₃M⁺, and M⁺ is a cation. In some embodiments, Q can be CH₂CH(OH)CH₂—SO₃H.

In some embodiments, y can be an integer from 11 to 60, (e.g., from 11 to 20, from 11 to 25, from 11 to 30, from 11 to 35, from 11 to 40, from 11 to 45, from 11 to 50, from 11 to 55, from 15 to 60, from 15 to 25, from 15 to 30, from 15 to 35, from 15 to 40, from 15 to 50, from 15 to 60, from 20 to 30, from 20 to 35, from 20 to 40, from 20 to 50, from 20 to 60, from 25 to 30, from 25 to 40, from 25 to 50, from 25 to 60, from 30 to 40, from 30 to 45, from 30 to 50, from 30 to 60, from 40 to 50, from 40 to 60, or from 50 to 40). In some embodiments, y can be 11. In some embodiments, y can be 15. In some embodiments, y can be 20. In some embodiments, y can be 25.

In some embodiments, x can be an integer from 0 to 15 (e.g., from 0 to 11, from 0 to 12, from 0 to 13, from 0 to 14, from 0 to 10, from 0 to 9, from 0 to 8, from 0 to 7, from 0 to 6, from 0 to 5, from 0 to 4, from 0 to 3, from 0 to 2, from 0 to 1, from 1 to 15, from 2 to 15, from 3 to 15, from 4 to 15, from 5 to 15, from 6 to 15, from 7 to 15, from 8 to 15, from 9 to 15, from 10 to 15, from 11 to 15, from 12 to 15, from 13 to 15, from 14 to 15, from 1 to 12, from 2 to 12, from 3 to 12, from 4 to 12, from 5 to 12, from 6 to 12, from 7 to 12, from 8 to 12, from 9 to 12, from 10 to 12, from 11 to 12, from 1 to 10, from 2 to 10, from 3 to 10, from 4 to 10, from 5 to 10, from 6 to 10, from 7 to 10, from 8 to 10, from 9 to 10, from 1 to 9, from 2 to 9, from 3 to 9, from 4 to 9, from 5 to 9, from 6 to 9, from 7 to 9, from 8 to 9, from 1 to 8, from 2 to 8, from 3 to 8, from 4 to 8, from 5 to 8, from 6 to 8, from 7 to 8, from 1 to 7, from 2 to 7, from 3 to 7, from 4 to 7, from 5 to 7, from 6 to 7, from 1 to 6, from 2 to 6, from 3 to 6, from 4 to 6, from 5 to 6, from 1 to 5, from 2 to 5, from 3 to 5, from 4 to 5, from 1 to 4, from 2 to 4, from 3 to 4, from 1 to 3, from 2 to 3, or from 1 to 2). In some embodiments, x can be 0, 1, 2, 3, 4, 5, 6, 7, 8, 9, 10, 11, 12, 13, 14, or 15. In some embodiments, x can be 0.

In some embodiments, z can be an integer from 0 to 100 (e.g. from 0 to 90, from 1 to 90, from 5 to 90, from 10 to 90, from 15 to 90, from 20 to 90, from 25 to 90, from 30 to 90, from 40 to 90, from 50 to 90, from 60 to 90, from 70 to 90, from 80 to 90, from 0 to 80, from 1 to 80, from 5 to 80, from 10 to 80, from 15 to 80, from 20 to 80, from 25 to 80, from 30 to 80, from 40 to 80, from 50 to 80, from 60 to 80, from 70 to 80, from 0 to 70, from 1 to 70, from 5 to 70, from 10 to 70, from 15 to 70, from 20 to 70, from 25 to 70, from 30 to 70, from 40 to 70, from 50 to 70, from 0 to 50, from 1 to 50, from 5 to 50, from 10 to 50, from 15 to 50, from 20 to 50, from 25 to 50, from 30 to 50, from 40 to 50, from 0 to 40, from 1 to 40, from 5 to 40, from 10 to 40, from 15 to 40, from 20 to 40, from 25 to 40, from 30 to 40, from 0 to 35, from 1 to 35, from 5 to 35, from 10 to 35, from 15 to 35, from 20 to 35, from 25 to 35, from 0 to 30, from 1 to 30, from 5 to 30, from 10 to 30, from 15 to 30, from 20 to 30, from 25 to 30, from 0 to 25, from 1 to 25, from 5 to 25, from 10 to 25, from 15 to 25, from 20 to 25, from 0 to 20, from 1 to 20, from 5 to 20, from 10 to 20, from 15 to 20, from 0 to 15, from 1 to 15, from 5 to 15, from 10 to 15, from 0 to 10, from 1 to 10, from 5 to 10, from 0 to 5, from 1 to 5, from 2 to 5, from 3 to 5, from 4 to 5, from 0 to 4, from 1 to 4, from 2 to 4, from 3 to 4, from 0 to 3, from 1 to 3, from 2 to 3, from 0 to 2, from 1 to 2, from 0 to 1). In some embodiments, z can be 0, 5, 10, 15, 20, 25, 30, 35, 40, 45, 50, 55, 60, 65, 70, 75, 80, 85, 90, 95, or 100. In some embodiments, z can be 0.

In some embodiments, z is greater than 1. In some embodiments, z is greater than 5, greater than 10, greater than 15, greater than 20, greater than 25, greater than 30, greater than 35, greater than 40, greater than 45, greater than 50, greater than 55, greater than 60, greater than 65, greater than 70, greater than 75, greater than 80, greater than 85, greater than 90, or greater than 95. In some embodiments, when Q is hydrogen, z can be at least 6 (e.g., at least 8, at least 10, at least 15, at least 20, at least 30, at least 45, at least 50, at least 55, at least 60, at least 65, at least 70, at least 75, at least 80, at least 85, at least 90, or at least 95).

In some embodiments, the surfactant can be an amine based polyol alkoxylate surfactant. The amine based polyol alkoxylate surfactant can be defined by Formula J

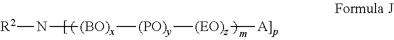

wherein
BO represents —CH₂—CH(ethyl)-O— or —CH₃CH(O—)CH₃;
PO represents —CH₂—CH(methyl)-O—;
EO represents —CH₂—CH₂—O—;
R² is absent, -((BO)ₓ-(PO)_{y}-(EO)_{z}-)ₘ-A, or —CH₂R³;
R³ is C₁-C₁₀ alkoxy, aryloxy, —C(O)O⁻M⁺, or —C(O)OH;
m is an integer from 1 to 3;
p is an integer from 2 to 3;
A is H, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
x is an integer from 0 to 15;
y is an integer from 11 to 60; and
z is an integer from 0 to 100,
wherein when R² is absent, p is 3.

In some embodiments, A can be H. In some embodiments, A can be —SO₃M⁺, —SO₃H, —CH₂C(O)O⁻M⁺, or —CH₂C(O)OH; and M⁺ is a cation. In some embodiments, A can be —SO₃M⁺; and M⁺ is a cation. In some embodiments, A can be-CH₂C(O)O⁻M⁺; and M⁺ is a cation. In some embodiments, A can be —SO₃H. In some embodiments, A can be-CH₂C(O)OH. In some embodiments, Q can be CH₂CH(OH)CH₂—SO₃M⁺, and M⁺ is a cation. In some embodiments, Q can be CH₂CH(OH)CH₂—SO₃H.

In some embodiments, y can be an integer from 11 to 60, (e.g., from 11 to 20, from 11 to 25, from 11 to 30, from 11 to 35, from 11 to 40, from 11 to 45, from 11 to 50, from 11 to 55, from 15 to 60, from 15 to 25, from 15 to 30, from 15 to 35, from 15 to 40, from 15 to 50, from 15 to 60, from 20 to 30, from 20 to 35, from 20 to 40, from 20 to 50, from 20 to 60, from 25 to 30, from 25 to 40, from 25 to 50, from 25 to 60, from 30 to 40, from 30 to 45, from 30 to 50, from 30 to 60, from 40 to 50, from 40 to 60, or from 50 to 40). In some embodiments, y can be 11. In some embodiments, y can be 15. In some embodiments, y can be 20. In some embodiments, y can be 25.

In some embodiments, x can be an integer from 0 to 15 (e.g., from 0 to 11, from 0 to 12, from 0 to 13, from 0 to 14, from 0 to 10, from 0 to 9, from 0 to 8, from 0 to 7, from 0 to 6, from 0 to 5, from 0 to 4, from 0 to 3, from 0 to 2, from 0 to 1, from 1 to 15, from 2 to 15, from 3 to 15, from 4 to 15, from 5 to 15, from 6 to 15, from 7 to 15, from 8 to 15, from 9 to 15, from 10 to 15, from 11 to 15, from 12 to 15, from 13 to 15, from 14 to 15, from 1 to 12, from 2 to 12, from 3 to 12, from 4 to 12, from 5 to 12, from 6 to 12, from 7 to 12, from 8 to 12, from 9 to 12, from 10 to 12, from 11 to 12, from 1 to 10, from 2 to 10, from 3 to 10, from 4 to 10, from 5 to 10, from 6 to 10, from 7 to 10, from 8 to 10, from 9 to 10, from 1 to 9, from 2 to 9, from 3 to 9, from 4 to 9, from 5 to 9, from 6 to 9, from 7 to 9, from 8 to 9, from 1 to 8, from 2 to 8, from 3 to 8, from 4 to 8, from 5 to 8, from 6 to 8, from 7 to 8, from 1 to 7, from 2 to 7, from 3 to 7, from 4 to 7, from 5 to 7, from 6 to 7, from 1 to 6, from 2 to 6, from 3 to 6, from 4 to 6, from 5 to 6, from 1 to 5, from 2 to 5, from 3 to 5, from 4 to 5, from 1 to 4, from 2 to 4, from 3 to 4, from 1 to 3, from 2 to 3, or from 1 to 2). In some embodiments, x can be 0, 1, 2, 3, 4, 5, 6, 7, 8, 9, 10, 11, 12, 13, 14, or 15. In some embodiments, x can be 0.

In some embodiments, z can be an integer from 0 to 100 (e.g. from 0 to 90, from 1 to 90, from 5 to 90, from 10 to 90, from 15 to 90, from 20 to 90, from 25 to 90, from 30 to 90, from 40 to 90, from 50 to 90, from 60 to 90, from 70 to 90, from 80 to 90, from 0 to 80, from 1 to 80, from 5 to 80, from 10 to 80, from 15 to 80, from 20 to 80, from 25 to 80, from 30 to 80, from 40 to 80, from 50 to 80, from 60 to 80, from 70 to 80, from 0 to 70, from 1 to 70, from 5 to 70, from 10 to 70, from 15 to 70, from 20 to 70, from 25 to 70, from 30 to 70, from 40 to 70, from 50 to 70, from 0 to 50, from 1 to 50, from 5 to 50, from 10 to 50, from 15 to 50, from 20 to 50, from 25 to 50, from 30 to 50, from 40 to 50, from 0 to 40, from 1 to 40, from 5 to 40, from 10 to 40, from 15 to 40, from 20 to 40, from 25 to 40, from 30 to 40, from 0 to 35, from 1 to 35, from 5 to 35, from 10 to 35, from 15 to 35, from 20 to 35, from 25 to 35, from 0 to 30, from 1 to 30, from 5 to 30, from 10 to 30, from 15 to 30, from 20 to 30, from 25 to 30, from 0 to 25, from 1 to 25, from 5 to 25, from 10 to 25, from 15 to 25, from 20 to 25, from 0 to 20, from 1 to 20, from 5 to 20, from 10 to 20, from 15 to 20, from 0 to 15, from 1 to 15, from 5 to 15, from 10 to 15, from 0 to 10, from 1 to 10, from 5 to 10, from 0 to 5, from 1 to 5, from 2 to 5, from 3 to 5, from 4 to 5, from 0 to 4, from 1 to 4, from 2 to 4, from 3 to 4, from 0 to 3, from 1 to 3, from 2 to 3, from 0 to 2, from 1 to 2, from 0 to 1). In some embodiments, z can be 0, 5, 10, 15, 20, 25, 30, 35, 40, 45, 50, 55, 60, 65, 70, 75, 80, 85, 90, 95, or 100. In some embodiments, z can be 0.

In some embodiments, z is greater than 1. In some embodiments, z is greater than 5, greater than 10, greater than 15, greater than 20, greater than 25, greater than 30, greater than 35, greater than 40, greater than 45, greater than 50, greater than 55, greater than 60, greater than 65, greater than 70, greater than 75, greater than 80, greater than 85, greater than 90, or greater than 95. In some embodiments, when Q is hydrogen, z can be at least 6 (e.g., at least 8, at least 10, at least 15, at least 20, at least 30, at least 45, at least 50, at least 55, at least 60, at least 65, at least 70, at least 75, at least 80, at least 85, at least 90, or at least 95).

In some embodiments, R² can be —CH₂R³. In some embodiments, R² is absent. In some embodiments, R² is -((BO)ₓ-(PO)_{y}-(EO)_{z}-)ₘ-A. In some embodiments, R³ is —C(O)O⁻M⁺, or —C(O)OH. In some embodiments, R³ is C₁-C₁₀ alkoxy. In some embodiments, R³ is aryloxy. In some embodiments, p is 3. In some embodiments, p is 2. In some embodiments, m is 2.

In some embodiments, the compound of Formula J can be selected from:

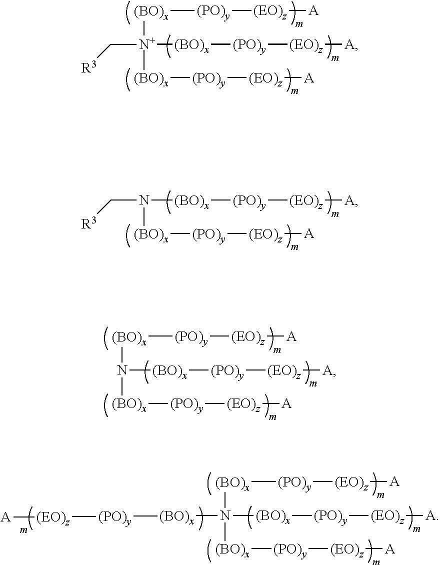

In some embodiments, the compound of Formula J can be selected from:

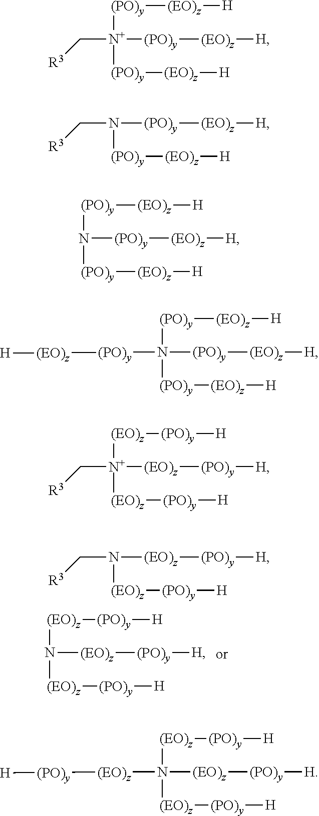

Suitable surfactants are disclosed, for example, in U.S. Pat. Nos. 3,811,504, 3,811,505, 3,811,507, 3,890,239, 4,463,806, 6,022,843, 6,225,267, and 7,629,299; International Patent Application Publication Nos. WO/2008/079855, WO/2012/027757, WO/2016/145164, and WO/2011/094442; as well as U.S. Patent Application Publication Nos. 2005/0199395, 2006/0185845, 2006/018486, 2009/0270281, 2011/0046024, 2011/0100402, 2011/0190175, 2007/0191633, 2010/004843.2011/0201531, 2011/0190174, 2011/0071057, 2011/0059873, 2011/0059872, 2011/0048721, 2010/0319920, 2010/0292110, and 2013/0281327, all of which are incorporated herein by reference in their entirety. Additional suitable surfactants are surfactants known to be used in enhanced oil recovery methods, including those discussed in D. B. Levitt, A. C. Jackson, L. Britton and G. A. Pope, “Identification and Evaluation of High-Performance FOR Surfactants,” SPE IX89, conference contribution for the SPE Symposium on Improved Oil Recovery Annual Meeting, Tulsa, Okla., Apr. 24-26, 2006.

A person having ordinary skill in the art will immediately recognize that many surfactants are commercially available as blends of related molecules (e.g., IOS and ABS surfactants). Thus, where a surfactant is present within a composition provided herein, a person of ordinary skill would understand that the surfactant might be a blend of a plurality of related surfactant molecules (as described herein and as generally known in the art).

In some embodiments, the total surfactant concentration (i.e., the compound of Formula I and one or more surfactants within the aqueous compositions provided herein) is from about 0.05% w/w to about 10% w/w. In other embodiments, the total surfactant concentration in the aqueous composition is from about 0.25% w/w to about 10% w/w. In other embodiments, the total surfactant concentration in the aqueous composition is about 0.5% w/w. In other embodiments, the total surfactant concentration in the aqueous composition is about 1.0% w/w. In other embodiments, the total surfactant concentration in the aqueous composition is about 1.25% w/w. In other embodiments, the total surfactant concentration in the aqueous composition is about 1.5% w/w. In other embodiments, the total surfactant concentration in the aqueous composition is about 1.75% w/w. In other embodiments, the total surfactant concentration in the aqueous composition is about 2.0% w/w. In other embodiments, the total surfactant concentration in the aqueous composition is about 2.5% w/w. In other embodiments, the total surfactant concentration in the aqueous composition is about 3.0% w/w. In other embodiments, the total surfactant concentration in the aqueous composition is about 3.5% w/w. In other embodiments, the total surfactant concentration in the aqueous composition is about 4.0% w/w. In other embodiments, the total surfactant concentration in the aqueous composition is about 4.5% w/w. In other embodiments, the total surfactant concentration in the aqueous composition is about 5.0% w/w. In other embodiments, the total surfactant concentration in the aqueous composition is about 5.5% w/w. In other embodiments, the total surfactant concentration in the aqueous composition is about 6.0% w/w. In other embodiments, the total surfactant concentration in the aqueous composition is about 6.5% w/w. In other embodiments, the total surfactant concentration in the aqueous composition is about 7.0% w/w. In other embodiments, the total surfactant concentration in the aqueous composition is about 7.5% w/w. In other embodiments, the total surfactant concentration in the aqueous composition is about 8.0% w/w. In other embodiments, the total surfactant concentration in the aqueous composition is about 9.0% w/w. In other embodiments, the total surfactant concentration in the aqueous composition is about 10% w/w.

In some embodiments, the concentration of the compound of Formula I is about 0.1%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.15%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.20%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.25%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.30%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.35%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.40%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.45%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.50%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.55%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.60%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.65%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.70%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.75%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.80%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.85%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.90%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.95%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 1.0%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 1.25%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 1.50%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 1.75%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 2%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 3%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 4%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 5%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the aqueous compositions can further include a viscosity enhancing water-soluble polymer. In some embodiments, the water-soluble polymer may be a biopolymer such as xanthan gum or scleroglucan, a synthetic polymer such as polyacryamide, hydrolyzed polyarcrylamide or co-polymers of acrylamide and acrylic acid, 2-acrylamido 2-methyl propane sulfonate or N-vinyl pyrrolidone, a synthetic polymer such as polyethylene oxide, or any other high molecular weight polymer soluble in water or brine. In some embodiments, the polymer is polyacrylamide (PAM), partially hydrolyzed polyacrylamides (HPAM), and copolymers of 2-acrylamido-2-methylpropane sulfonic acid or sodium salt or mixtures thereof, and polyacrylamide (PAM) commonly referred to as AMPS copolymer and mixtures of the copolymers thereof. In one embodiment, the viscosity enhancing water-soluble polymer is polyacrylamide or a co-polymer of polyacrylamide. In one embodiment, the viscosity enhancing water-soluble polymer is a partially (e.g. 20%, 25%, 30%, 35%, 40%, 45%) hydrolyzed anionic polyacrylamide. In some further embodiment, the viscosity enhancing water-soluble polymer has a molecular weight of approximately about 8×10⁶ Daltons. In some other further embodiment, the viscosity enhancing water-soluble polymer has a molecular weight of approximately about 18×10⁶ Daltons. Non-limiting examples of commercially available polymers useful for the invention including embodiments provided herein are Florpaam 3330S and Florpaam 3360S. Molecular weights of the polymers may range from about 10,000 Daltons to about 20,000,000 Daltons. In some embodiments, the viscosity enhancing water-soluble polymer is used in the range of about 500 to about 5000 ppm concentration, such as from about 1000 to 2000 ppm (e.g., in order to match or exceed the reservoir oil viscosity under the reservoir conditions of temperature and pressure).

In some embodiments, the aqueous compositions can further include an alkali agent. An alkali agent as provided herein can be a basic, ionic salt of an alkali metal (e.g., lithium, sodium, potassium) or alkaline earth metal element (e.g., magnesium, calcium, barium, radium). Examples of suitable alkali agents include, for example, NaOH, KOH, LiOH, Na₂CO₃, NaHCO₃, Na-metaborate, Na silicate, Na orthosilicate, Na acetate or NH₄OH. The aqueous composition may include seawater, or fresh water from an aquifer, river or lake. In some embodiments, the aqueous composition includes hard brine water or soft brine water. In some further embodiments, the water is soft brine water. In some further embodiments, the water is hard brine water. Where the aqueous composition includes soft brine water, the aqueous composition can further include an alkaline agent. In soft brine water the alkaline agent can provide for enhanced soap generation from the active oils, lower surfactant adsorption to the solid material (e.g., rock) in the reservoir and increased solubility of viscosity enhancing water soluble polymers.

The alkali agent can be present in the aqueous composition at a concentration from about 0.1% w/w to about 10% w/w. The combined amount of alkali agent and compound provided herein (e.g., compound of Formula I) present in the aqueous composition provided herein can be approximately equal to or less than about 10% w/w. In some embodiments, the total concentration of alkali agent (i.e., the total amount of alkali agent within the aqueous compositions and emulsion compositions provided herein) in is from about 0.05% w/w to about 5% w/w. In other embodiments, the total alkali agent concentration in the aqueous composition is from about 0.25% w/w to about 5% w/w. In other embodiments, the total alkali agent concentration in the aqueous composition is about 0.5% w/w. In other embodiments, the total alkali agent concentration in the aqueous composition is about 0.75% w/w. In other embodiments, the total alkali agent concentration in the aqueous composition is about 1% w/w. In other embodiments, the total alkali agent concentration in the aqueous composition is about 1.25% w/w. In other embodiments, the total alkali agent concentration in the aqueous composition is about 1.50% w/w. In other embodiments, the total alkali agent concentration in the aqueous composition is about 1.75% w/w. In other embodiments, the total alkali agent concentration in the aqueous composition is about 2% w/w. In other embodiments, the total alkali agent concentration in the aqueous composition is about 2.25% w/w. In other embodiments, the total alkali agent concentration in the aqueous composition is about 2.5% w/w. In other embodiments, the total alkali agent concentration in the aqueous composition is about 2.75% w/w. In other embodiments, the total alkali agent concentration in the aqueous composition is about 3% w/w. In other embodiments, the total alkali agent concentration in the aqueous composition is about 3.25% w/w. In other embodiments, the total alkali agent concentration in the aqueous composition is about 3.5% w/w. In other embodiments, the total alkali agent concentration in the aqueous composition is about 3.75% w/w. In other embodiments, the total alkali agent concentration in the aqueous composition is about 4% w/w. In other embodiments, the total alkali agent concentration in the aqueous composition is about 4.25% w/w. In other embodiments, the total alkali agent concentration in the aqueous composition is about 4.5% w/w. In other embodiments, the total alkali agent concentration in the aqueous composition is about 4.75% w/w. In other embodiments, the total alkali agent concentration in the aqueous composition is about 5.0% w/w. In some embodiments, the alkali agent can be present in the aqueous compositions in an effective amount to afford an aqueous composition having a pH of from 10 to 12 (e.g., 10.5 to 11.5).

In some embodiments, the aqueous compositions can further include a co-solvent. In embodiments, the co-solvent is an alcohol, alcohol ethoxylate, glycol ether, glycols, or glycerol. The aqueous compositions provided herein may include more than one co-solvent. Thus, in embodiments, the aqueous composition includes a plurality of different co-solvents. Where the aqueous composition includes a plurality of different co-solvents, the different co-solvents can be distinguished by their chemical (structural) properties. For example, the aqueous composition may include a first co-solvent, a second co-solvent and a third co-solvent, wherein the first co-solvent is chemically different from the second and the third co-solvent, and the second co-solvent is chemically different from the third co-solvent. In embodiments, the plurality of different co-solvents includes at least two different alcohols (e.g., a C₁-C₆ alcohol and a C₁-C₄ alcohol). In embodiments, the aqueous composition includes a C₁-C₆ alcohol and a C₁-C₄ alcohol. In embodiments, the plurality of different co-solvents includes at least two different alkoxy alcohols (e.g., a C₁-C₆ alkoxy alcohol and a C₁-C₄ alkoxy alcohol). In embodiments, the aqueous composition includes a C₁-C₆ alkoxy alcohol and a C₁-C₄ alkoxy alcohol. In embodiments, the plurality of different co-solvents includes at least two co-solvents selected from the group consisting of alcohols, alkyl alkoxy alcohols and phenyl alkoxy alcohols. For example, the plurality of different co-solvents may include an alcohol and an alkyl alkoxy alcohol, an alcohol and a phenyl alkoxy alcohol, or an alcohol, an alkyl alkoxy alcohol and a phenyl alkoxy alcohol. The alkyl alkoxy alcohols or phenyl alkoxy alcohols provided herein have a hydrophobic portion (alkyl or aryl chain), a hydrophilic portion (e.g., an alcohol) and optionally an alkoxy (ethoxylate or propoxylate) portion. Thus, in embodiments, the co-solvent is an alcohol, alkoxy alcohol, glycol ether, glycol or glycerol. Suitable co-solvents are known in the art, and include, for example, surfactants described in U.S. Patent Application Publication No. 2013/0281327 which is hereby incorporated herein in its entirety.

In some embodiments, the co-solvent can comprise a polyol alkoxylate co-solvent. Suitable polyol alkoxylate co-solvents include those defined by Formula I below

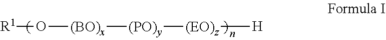

wherein
BO represents —CH₂—CH(ethyl)-O— or —CH₃CH(O—)CH₃;
PO represents —CH₂—CH(methyl)-O—;
EO represents —CH₂—CH₂—O—;
R¹ represents a C₁-C₈ alkyl group (e.g., a C₁-C₆ alkyl group) or a cyclic structure derived from alkoxylation of an alkyl monoglucoside, an alkyl polyglucoside, a monosaccharide, a disaccharide, or a polysaccharide;
n is an integer from 2 to 6;
x is an integer from 0 to 5;
y is an integer from 1 to 10; and
z is an integer from 0 to 50.

In some embodiments, n can be an integer from 3 to 6, from 3 to 5, from 3 to 4, from 4 to 5, or from 4 to 6. In some embodiments, n can be an integer from 3 to 6. In some embodiments, n can be 3, 4, 5, or 6. In some embodiments, n is 3.

In some embodiments, R¹ can be a C₁-C₆ alkyl, C₁-C₅ alkyl, C₁-C₄ alkyl, C₁-C₃ alkyl, C₁-C₂ alkyl, C₂-C₆ alkyl, C₂-C₅ alkyl, C₂-C₄ alkyl, C₂-C₃ alkyl, C₃-C₆ alkyl, C₃-C₅ alkyl, C₃-C₄ alkyl, C₄-C₅ alkyl, C₄-C₆ alkyl, or C₅-C₆ alkyl group. In some embodiments, R¹ can be a C₃-C₆ alkyl group.

In other embodiments, R¹ can comprise a monocyclic or polycyclic structure derived from alkoxylation of an alkyl monoglucoside, an alkyl polyglucoside, a monosaccharide, a disaccharide, or a polysaccharide.

In some embodiments, the compound of Formula I can be defined by Formula II below

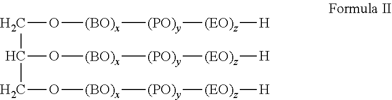

wherein
BO represents —CH₂—CH(ethyl)-O— or —CH₃CH(O—)CH₃;
PO represents —CH₂—CH(methyl)-O—;
EO represents —CH₂—CH₂—O—;
x is an integer from 0 to 5;
y is an integer from 1 to 10; and
z is an integer from 0 to 50.

In some embodiments, the compound of Formula I can be defined by Formula III below

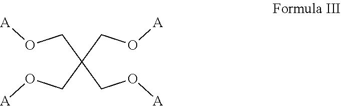

wherein
A represents -(BO)ₓ-(PO)_{y}-(EO)_{z}-H
BO represents —CH₂—CH(ethyl)-O— or —CH₃CH(O—)CH₃;
PO represents —CH₂—CH(methyl)-O—;
EO represents —CH₂—CH₂—O—;
x is an integer from 0 to 5;
y is an integer from 1 to 10; and
z is an integer from 0 to 50.

In some embodiments, the compound of Formula I can be defined by Formula III-a below

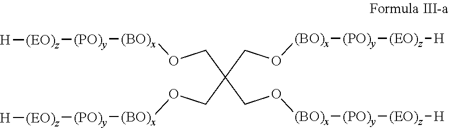

wherein
BO represents —CH₂—CH(ethyl)-O— or —CH₃CH(O—)CH₃;
PO represents —CH₂—CH(methyl)-O—;
EO represents —CH₂—CH₂—O—;
x is an integer from 0 to 5;
y is an integer from 1 to 10; and
z is an integer from 0 to 50.

In some embodiments, the compound of Formula I can be defined by Formula IV below

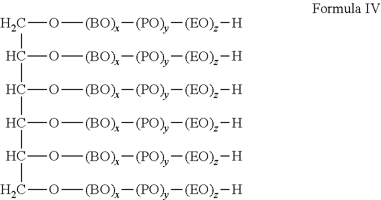

wherein
BO represents —CH₂—CH(ethyl)-O— or —CH₃CH(O—)CH₃;
PO represents —CH₂—CH(methyl)-O—;
EO represents —CH₂—CH₂—O—;
x is an integer from 0 to 5;
y is an integer from 1 to 10; and
z is an integer from 0 to 50.

In some embodiments, the compound of Formula I can be defined by Formula V below

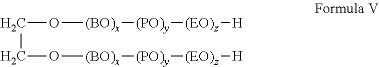

wherein
BO represents —CH₂—CH(ethyl)-O— or —CH₃CH(O—)CH₃;
PO represents —CH₂—CH(methyl)-O—;
EO represents —CH₂—CH₂—O—;
x is an integer from 0 to 5;
y is an integer from 1 to 10; and
z is an integer from 0 to 50.

In some embodiments, the compound of Formula I can be defined of Formula VI below

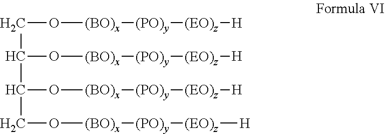

wherein
BO represents —CH₂—CH(ethyl)-O— or —CH₃CH(O—)CH₃;
PO represents —CH₂—CH(methyl)-O—;
EO represents —CH₂—CH₂—O—;
x is an integer from 0 to 5;
y is an integer from 1 to 10; and
z is an integer from 0 to 50.

In some embodiments, the compound of Formula I can be defined of Formula VII below

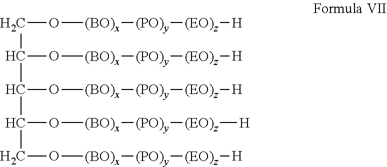

wherein
BO represents —CH₂—CH(ethyl)-O— or —CH₃CH(O—)CH₃;
PO represents —CH₂—CH(methyl)-O—;
EO represents —CH₂—CH₂—O—;
x is an integer from 0 to 5;
y is an integer from 1 to 10; and
z is an integer from 0 to 50.

In some embodiments, the compound of Formula I can be defined by Formula VIII below

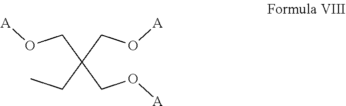

wherein
A represents -(BO)ₓ-(PO)_{y}-(EO)_{z}-H
BO represents —CH₂—CH(ethyl)-O— or —CH₃CH(O—)CH₃;
PO represents —CH₂—CH(methyl)-O—;
EO represents —CH₂—CH₂—O—;
x is an integer from 0 to 15;
y is an integer from 1 to 10; and
z is an integer from 0 to 50.

In some embodiments, the compound of Formula I can be defined by Formula IV-a below

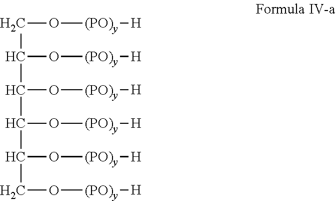

wherein
PO represents —CH₂—CH(methyl)-O—; and
y is an integer from 1 to 10.

In some embodiments, the compound of Formula I can be defined by Formula V-a below

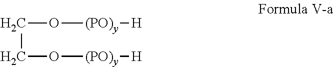

wherein
PO represents —CH₂—CH(methyl)-O—; and
y is an integer from 1 to 10.

In some embodiments, the compound of Formula I can be defined by Formula II-a below

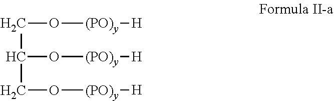

wherein
PO represents —CH₂—CH(methyl)-O—; and
y is an integer from 1 to 10.

In some embodiments, the compound of Formula I can be defined by Formula VI-a below

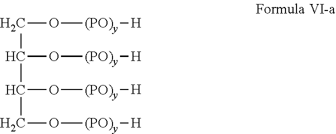

wherein
PO represents —CH₂—CH(methyl)-O—; and
y is an integer from 1 to 10.

In some embodiments, the compound of Formula I can be defined by Formula VII-a below

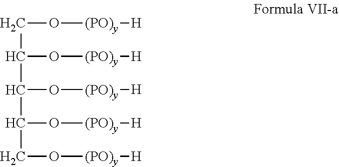

wherein
PO represents —CH₂—CH(methyl)-O—; and
y is an integer from 1 to 10.

In some embodiments, the compound of Formula I can be defined by Formula VIII below

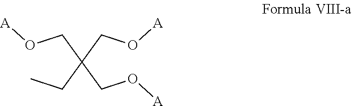

wherein
A represents -(PO)_{y}-H
PO represents —CH₂—CH(methyl)-O—; and
y is an integer from 1 to 10.

In some embodiments, the compound of Formula I can be defined by Formula II-b below

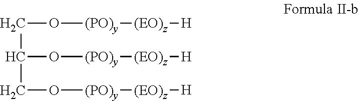

wherein
PO represents —CH₂—CH(methyl)-O—;
EO represents —CH₂—CH₂—O—;
y is an integer from 1 to 10; and
z is an integer from 0 to 50.

In some embodiments, the compound of Formula I can be defined by Formula III-b below

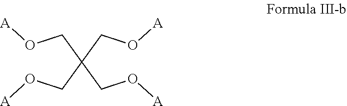

wherein
A represents -(PO)_{y}-(EO)_{z}-H
PO represents —CH₂—CH(methyl)-O—;
EO represents —CH₂—CH₂—O—;
y is an integer from 1 to 10; and
z is an integer from 0 to 50.

In some embodiments, the compound of Formula I can be defined by Formula III-c below

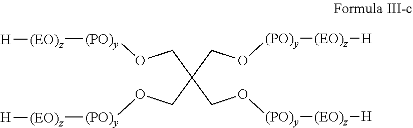

wherein
PO represents —CH₂—CH(methyl)-O—;
EO represents —CH₂—CH₂—O—;
y is an integer from 1 to 10; and
z is an integer from 0 to 50.

In some embodiments, the compound of Formula I can be defined by Formula IV-b below

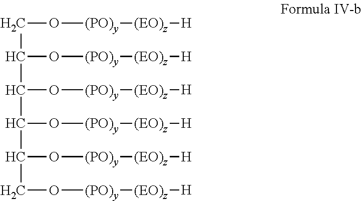

wherein
PO represents —CH₂—CH(methyl)-O—;
EO represents —CH₂—CH₂—O—;
y is an integer from 1 to 10; and
z is an integer from 0 to 50.

In some embodiments, the compound of Formula I can be defined by Formula V-b below

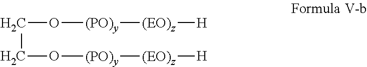

wherein
PO represents —CH₂—CH(methyl)-O—;
EO represents —CH₂—CH₂—O—;
y is an integer from 1 to 10; and
z is an integer from 0 to 50.

In some embodiments, the compound of Formula I can be defined of Formula VI-b below

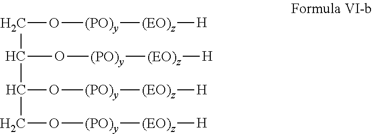

wherein
PO represents —CH₂—CH(methyl)-O—;
EO represents —CH₂—CH₂—O—;
y is an integer from 1 to 10; and
z is an integer from 0 to 50.

In some embodiments, the compound of Formula I can be defined of Formula VII-b below

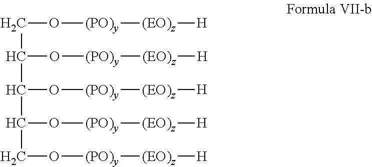

wherein
PO represents —CH₂—CH(methyl)-O—;
EO represents —CH₂—CH₂—O—;
y is an integer from 1 to 10; and
z is an integer from 0 to 50.

In some embodiments, the compound of Formula I can be defined by Formula VIII-b below

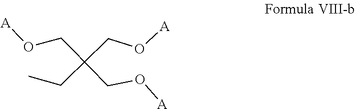

wherein
A represents -(PO)_{y}-(EO)_{z}-H
PO represents —CH₂—CH(methyl)-O—;
EO represents —CH₂—CH₂—O—;
y is an integer from 1 to 10; and
z is an integer from 0 to 50.

In some embodiments, the compound of Formula I can be defined by Formula II-c below

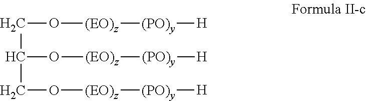

wherein
PO represents —CH₂—CH(methyl)-O—;
EO represents —CH₂—CH₂—O—;
y is an integer from 1 to 10; and
z is an integer from 0 to 50.

In some embodiments, the compound of Formula I can be defined by Formula III-c below

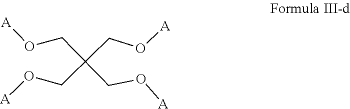

wherein
A represents -(EO)_{z}-(PO)_{y}-A
PO represents —CH₂—CH(methyl)-O—;
EO represents —CH₂—CH₂—O—;
y is an integer from 1 to 10; and
z is an integer from 0 to 50.

In some embodiments, the compound of Formula I can be defined by Formula C-c below

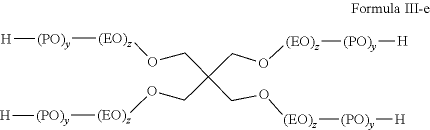

wherein
PO represents —CH₂—CH(methyl)-O—;
EO represents —CH₂—CH₂—O—;
y is an integer from 11 to 60; and
y is an integer from 1 to 10; and
z is an integer from 0 to 50.

In some embodiments, the compound of Formula I can be defined by Formula IV-c below

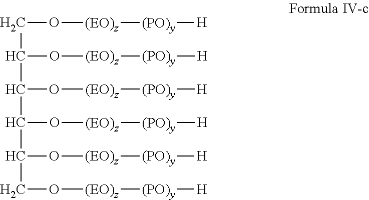

wherein
PO represents —CH₂—CH(methyl)-O—;
EO represents —CH₂—CH₂—O—;
y is an integer from 1 to 10; and
z is an integer from 0 to 50.

In some embodiments, the compound of Formula I can be defined by Formula V-c below

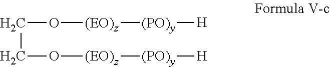

wherein
PO represents —CH₂—CH(methyl)-O—;
EO represents —CH₂—CH₂—O—;
y is an integer from 1 to 10; and
z is an integer from 0 to 50.

In some embodiments, the compound of Formula I can be defined of Formula VI-c below

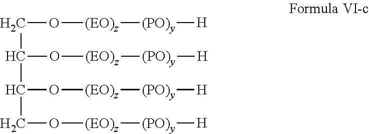

wherein
PO represents —CH₂—CH(methyl)-O—;
EO represents —CH₂—CH₂—O—;
y is an integer from 1 to 10; and
z is an integer from 0 to 50.

In some embodiments, the compound of Formula I can be defined of Formula VII-c below

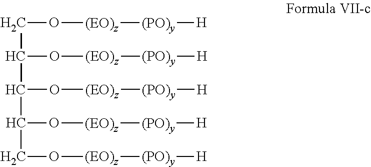

wherein
PO represents —CH₂—CH(methyl)-O—;
EO represents —CH₂—CH₂—O—;
y is an integer from 1 to 10; and
z is an integer from 0 to 50.

In some embodiments, the compound of Formula I can be defined by Formula VIII-c below

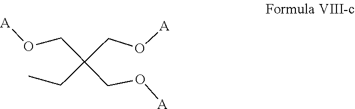

wherein
A represents -(EO)_{z}-(PO)_{y}-H
PO represents —CH₂—CH(methyl)-O—;
EO represents —CH₂—CH₂—O—;
R¹ represents a C₁-C₆ alkyl group;
y is an integer from 1 to 10; and
z is an integer from 0 to 50.

In some embodiments, y can be an integer from 1 to 10, (e.g., from 1 to 2, from 1 to 3, from 1 to 4, from 1 to 5, from 1 to 6, from 1 to 7, from 1 to 8, from 1 to 9, from 2 to 3, from 2 to 4, from 2 to 5, from 2 to 6, from 2 to 7, from 2 to 8, from 2 to 9, from 2 to 10, from 3 to 4, from 3 to 5, from 3 to 6, from 3 to 7, from 3 to 8, from 3 to 9, from 3 to 10, from 4 to 5, from 4 to 6, from 4 to 7, from 4 to 8, from 4 to 9, from 4 to 10, from 5 to 6, from 5 to 7, from 5 to 8, from 5 to 9, from 5 to 10, from 6 to 7, from 6 to 8, from 6 to 9, from 6 to 10, from 7 to 8, from 7 to 9, from 7 to 10, from 8 to 9, from 8 to 10, or from 9 to 10). In some embodiments, y can be 1. In some embodiments, y can be 4. In some embodiments, y can be 6. In some embodiments, y can be 10.

In some embodiments, x can be an integer from 0 to 5 (e.g., from 0 to 5, from 1 to 5, from 2 to 5, from 3 to 5, from 4 to 5, from 0 to 4, from 1 to 4, from 2 to 4, from 3 to 4, from 0 to 3, from 1 to 3, from 2 to 3, from 0 to 2, from 1 to 2, or from 0 to 1). In some embodiments, x can be 0, 1, 2, 3, 4, or 5. In some embodiments, x can be 0.

In some embodiments, z can be an integer from 0 to 50 (e.g. from 0 to 50, from 1 to 50, from 5 to 50, from 10 to 50, from 15 to 50, from 20 to 50, from 25 to 50, from 30 to 50, from 40 to 50, from 0 to 40, from 1 to 40, from 5 to 40, from 10 to 40, from 15 to 40, from 20 to 40, from 25 to 40, from 30 to 40, from 0 to 35, from 1 to 35, from 5 to 35, from 10 to 35, from 15 to 35, from 20 to 35, from 25 to 35, from 0 to 30, from 1 to 30, from 5 to 30, from 10 to 30, from 15 to 30, from 20 to 30, from 25 to 30, from 0 to 25, from 1 to 25, from 5 to 25, from 10 to 25, from 15 to 25, from 20 to 25, from 0 to 20, from 1 to 20, from 5 to 20, from 10 to 20, from 15 to 20, from 0 to 15, from 1 to 15, from 5 to 15, from 10 to 15, from 0 to 10, from 1 to 10, from 5 to 10, from 0 to 5, from 1 to 5, from 2 to 5, from 3 to 5, from 4 to 5, from 0 to 4, from 1 to 4, from 2 to 4, from 3 to 4, from 0 to 3, from 1 to 3, from 2 to 3, from 0 to 2, from 1 to 2, from 0 to 1). In some embodiments, z can be 0, 5, 10, 15, 20, 25, 30, 35, 40, 45, or 50. In some embodiments, z can be 0.

In some embodiments, z is greater than 1. In some embodiments, z is greater than 5, greater than 10, greater than 15, greater than 20, greater than 25, greater than 30, greater than 35, greater than 40, or greater than 45.

In some embodiments, the compound of Formula I can be defined by Formula IX below

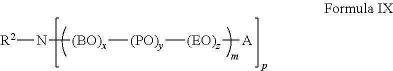

wherein
BO represents —CH₂—CH(ethyl)-O— or —CH₃CH(O—)CH₃;
PO represents —CH₂—CH(methyl)-O—;
EO represents —CH₂—CH₂—O—;
R² is absent, -((BO)ₓ-(PO)_{y}-(EO)_{z}-)ₘ-A, or —CH₂R³;
R³ is C₁-C₁₀ alkoxy, aryloxy, or —C(O)O⁻M⁺, or —C(O)OH;
m is an integer from 1 to 3;
p is an integer from 2 to 3;
A is H;
M⁺, when present, is a cation;
x is an integer from 0 to 5;
y is an integer from 1 to 10; and
z is an integer from 0 to 50,
wherein when R² is absent, p is 3.

In some embodiments, R² can be —CH₂R³. In some embodiments, R² is absent. In some embodiments, R² is —CH₂C(O)O⁻M⁺, or —CH₂C(O)OH.

In some embodiments, p is 3. In some embodiments, p is 2. In some embodiments, m is 2.

In some embodiments, the compound of Formula IX can be selected from:

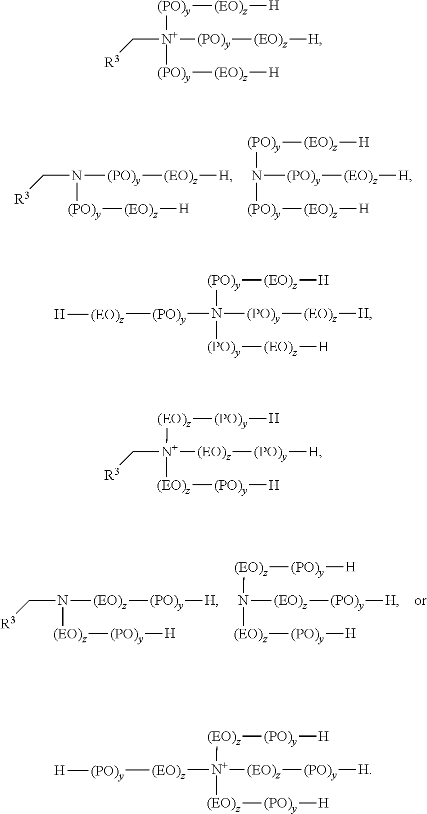

In some embodiments, y can be an integer from 1 to 10, (e.g., from 1 to 2, from 1 to 3, from 1 to 4, from 1 to 5, from 1 to 6, from 1 to 7, from 1 to 8, from 1 to 9, from 2 to 3, from 2 to 4, from 2 to 5, from 2 to 6, from 2 to 7, from 2 to 8, from 2 to 9, from 2 to 10, from 3 to 4, from 3 to 5, from 3 to 6, from 3 to 7, from 3 to 8, from 3 to 9, from 3 to 10, from 4 to 5, from 4 to 6, from 4 to 7, from 4 to 8, from 4 to 9, from 4 to 10, from 5 to 6, from 5 to 7, from 5 to 8, from 5 to 9, from 5 to 10, from 6 to 7, from 6 to 8, from 6 to 9, from 6 to 10, from 7 to 8, from 7 to 9, from 7 to 10, from 8 to 9, from 8 to 10, or from 9 to 10). In some embodiments, y can be 1. In some embodiments, y can be 4. In some embodiments, y can be 6. In some embodiments, y can be 10.

In some embodiments, x can be an integer from 0 to 5 (e.g., from 0 to 5, from 1 to 5, from 2 to 5, from 3 to 5, from 4 to 5, from 0 to 4, from 1 to 4, from 2 to 4, from 3 to 4, from 0 to 3, from 1 to 3, from 2 to 3, from 0 to 2, from 1 to 2, or from 0 to 1). In some embodiments, x can be 0, 1, 2, 3, 4, or 5. In some embodiments, x can be 0.

In some embodiments, z can be an integer from 0 to 50 (e.g. from 0 to 50, from 1 to 50, from 5 to 50, from 10 to 50, from 15 to 50, from 20 to 50, from 25 to 50, from 30 to 50, from 40 to 50, from 0 to 40, from 1 to 40, from 5 to 40, from 10 to 40, from 15 to 40, from 20 to 40, from 25 to 40, from 30 to 40, from 0 to 35, from 1 to 35, from 5 to 35, from 10 to 35, from 15 to 35, from 20 to 35, from 25 to 35, from 0 to 30, from 1 to 30, from 5 to 30, from 10 to 30, from 15 to 30, from 20 to 30, from 25 to 30, from 0 to 25, from 1 to 25, from 5 to 25, from 10 to 25, from 15 to 25, from 20 to 25, from 0 to 20, from 1 to 20, from 5 to 20, from 10 to 20, from 15 to 20, from 0 to 15, from 1 to 15, from 5 to 15, from 10 to 15, from 0 to 10, from 1 to 10, from 5 to 10, from 0 to 5, from 1 to 5, from 2 to 5, from 3 to 5, from 4 to 5, from 0 to 4, from 1 to 4, from 2 to 4, from 3 to 4, from 0 to 3, from 1 to 3, from 2 to 3, from 0 to 2, from 1 to 2, from 0 to 1). In some embodiments, z can be 0, 5, 10, 15, 20, 25, 30, 35, 40, 45, or 50. In some embodiments, z can be 0.

In some embodiments, z is greater than 1. In some embodiments, z is greater than 5, greater than 10, greater than 15, greater than 20, greater than 25, greater than 30, greater than 35, greater than 40, or greater than 45.

In some embodiments, a co-solvent can be present in an amount sufficient to increase the solubility of the alkoxylated co-solvent and/or surfactant (when present) in the aqueous phase relative to the absence of the co-solvent. In other words, in the presence of a sufficient amount of the co-solvent, the solubility of the co-solvent in the aqueous phase is higher than in the absence of the co-solvent. In embodiments, the co-solvent can be present in an amount sufficient to increase the solubility of the surfactant in the aqueous phase relative to the absence of the co-solvent. Thus, in the presence of a sufficient amount of the co-solvent the solubility of the surfactant in the aqueous phase can be higher than in the absence of the co-solvent. In embodiments, the co-solvent can be present in an amount sufficient to decrease the viscosity of an emulsion formed from the composition relative to the absence of the co-solvent.

In other embodiments, the aqueous composition can be substantially free of co-solvents (e.g., the composition can include less than 0.05% by weight co-solvents, based on the total weight of the composition).

In some embodiments, the aqueous composition can further include a gas. For instance, the gas may be combined with the aqueous composition to reduce its mobility by decreasing the liquid flow in the pores of the solid material (e.g., rock). In some embodiments, the gas may be supercritical carbon dioxide, nitrogen, natural gas or mixtures of these and other gases.

In some embodiments, the aqueous composition can have a pH of at least 7 (e.g., a pH of at least 7.5, a pH of at least 8, a pH of at least 8.5, a pH of at least 9, a pH of at least 9.5, a pH of at least 10, a pH of at least 10.5, a pH of at least 11, a pH of at least 11.5, or a pH of at least 12.5). In some embodiments, the aqueous composition can have a pH of 13 or less (e.g., a pH of 12.5 or less, a pH of 12 or less, a pH of 11.5 or less, a pH of 11 or less, a pH of 10.5 or less, a pH of 10 or less, a pH of 9.5 or less, a pH of 9 or less, a pH of 8.5 or less, a pH of 8 or less, or a pH of 7.5 or less).

The aqueous composition can have a pH ranging from any of the minimum values described above to any of the maximum values described above. For example, the aqueous composition can have a pH of from 7 to 13 (e.g., from 10 to 12, or from 10.5 to 11.5).

In some embodiments, the aqueous composition can have a salinity of at least 5,000 ppm. In other embodiments, the aqueous composition has a salinity of at least 50,000 ppm. In other embodiments, the aqueous composition has a salinity of at least 100,000 ppm. In other embodiments, the aqueous composition has a salinity of at least 250,000 ppm. The total range of salinity (total dissolved solids in the brine) is 100 ppm to saturated brine (about 260,000 ppm). The aqueous composition may include seawater, brine or fresh water from an aquifer, river or lake. The aqueous combination may further include salt to increase the salinity. In some embodiments, the salt is NaCl, KCl, CaCl₂), MgCl₂, CaSO₄, Na acetate or Na₂CO₃.

In some embodiments, the aqueous composition can have a temperature of at least 20° C. (e.g., at least 30° C., at least 40° C., at least 50° C., at least 60° C., at least 70° C., at least 80° C., at least 90° C., at least 100° C., or at least 110° C.). The aqueous composition can have a temperature of 120° C. or less (e.g., 110° C. or less, 100° C. or less, 90° C. or less, 80° C. or less, 70° C. or less, 60° C. or less, 50° C. or less, 40° C. or less, or 30° C. or less).

The aqueous composition can have a temperature ranging from any of the minimum values described above to any of the maximum values described above. For example, the aqueous composition can have a temperature of from 20° C. to 120° C. (e.g., from 50° C. to 120° C., or from 80° C. to 120° C.).

In some embodiments, the aqueous composition can have a viscosity of between 20 mPas and 100 mPas at 20° C. The viscosity of the aqueous solution may be increased from 0.3 mPas to 1, 2, 10, 20, 100 or even 1000 mPas by including a water-soluble polymer. As mentioned above, the apparent viscosity of the aqueous composition may be increased with a gas (e.g., a foam forming gas) as an alternative to the water-soluble polymer.

Also provided are emulsions comprising a polyol alkoxylate co-solvent or an aqueous composition described herein and unrefined petroleum. In some embodiments, the emulsion composition can be a microemulsion. A “microemulsion” as referred to herein is a thermodynamically stable mixture of oil, water and surfactants that may also include additional components such as additional co-solvents, electrolytes, alkali and polymers. In contrast, a “macroemulsion” as referred to herein is a thermodynamically unstable mixture of oil and water that may also include additional components. The emulsion composition provided herein may be an oil-in-water emulsion, wherein a surfactant forms aggregates (e.g., micelles) where the hydrophilic part of the surfactant molecule(s) contacts the aqueous phase of the emulsion and the lipophilic part contacts the oil phase of the emulsion. Thus, in some embodiments, the surfactant(s) form part of the aqueous part of the emulsion. And in other embodiments, the surfactant(s) form part of the oil phase of the emulsion. In yet another embodiment, the surfactant(s) form part of an interface between the aqueous phase and the oil phase of the emulsion.

In other embodiments, the oil and water solubilization ratios are insensitive to the combined concentration of divalent metal cations (e.g., Ca²⁺ and Mg²⁺) within the emulsion composition. In other embodiments, the oil and water solubilization ratios are insensitive to the salinity of the water or to all of the specific electrolytes contained in the water. The term “insensitive” used in the context of this paragraph means that the solubilization ratio tends not to change (e.g., tends to remain constant) as the concentration of divalent metal cations and/or salinity of water changes. In some embodiments, the change in the solubilization ratios are less than 5%, 10%, 20%, 30%, 40%, or 50% over a divalent metal cation concentration range of 10 ppm, 100 ppm, 1000 ppm or 10,000 ppm. In another embodiment, the change in the solubilization ratios are less than 5%, 10%, 20%, 30%, 40%, or 50% over a salinity concentration range of 10 ppm, 100 ppm, 1000 ppm or 10,000 ppm.

### Methods

In another aspect, a method of displacing a hydrocarbon material in contact with a solid material is provided. The method includes contacting a hydrocarbon material with a compound as described herein or the composition described herein, wherein the hydrocarbon material is in contact with a solid material. The hydrocarbon material is allowed to separate from the solid material thereby displacing the hydrocarbon material in contact with the solid material.

In some embodiments, the method can be a method for recovering hydrocarbons from a subterranean formation. The method including a) introducing the compound described herein or the composition described herein through a wellbore into the subterranean formation; and b) producing fluids from the subterranean formation; wherein the hydrocarbons in the subterranean formation comprise live oil. In some embodiments, step (a) comprises injecting the compound described herein or the composition described herein through an injection wellbore into the subterranean formation. In some embodiments, step (b) comprises producing the fluids from a production wellbore spaced apart from the injection wellbore a predetermined distance and in fluid communication with the subterranean formation. In some embodiments, the injection of the compound described herein or the composition described herein increases a flow of hydrocarbons to the production wellbore.

In some embodiments, the unrefined petroleum comprises live oil. In some embodiments, the live oil has a gas/oil ratio (GOR) of from 100 to 10,000 scf/bbl, such as from 200 to 10,000 scf/bbl, from 500 to 5,000 scf/bbl, from 500 to 1,500 scf/bbl, from 1,000 to 6,000 scf/bbl, or from 1,000 to 5,000 scf/bbl.

In other embodiments, the hydrocarbon material is unrefined petroleum (e.g., in a petroleum reservoir). In some further embodiments, the unrefined petroleum is an unrefined petroleum with an API gravity greater than 30. In some embodiments, the API gravity of the unrefined petroleum is greater than 30. In other embodiments, the API gravity of the unrefined petroleum is greater than 40. In some embodiments, the API gravity of the unrefined petroleum is greater than 50. In other embodiments, the API gravity of the unrefined petroleum is greater than 60. In some embodiments, the API gravity of the unrefined petroleum is greater than 70. In other embodiments, the API gravity of the unrefined petroleum is greater than 80. In some embodiments, the API gravity of the unrefined petroleum is greater than 90. In other embodiments, the API gravity of the unrefined petroleum is greater than 100. In some other embodiments, the API gravity of the unrefined petroleum is between 30 and 100.

The solid material may be a natural solid material (i.e., a solid found in nature such as rock). The natural solid material may be found in a petroleum reservoir. In some embodiments, the method is an enhanced oil recovery method. Enhanced oil recovery methods are well known in the art. A general treatise on enhanced oil recovery methods is Basic Concepts in Enhanced Oil Recovery Processes edited by M. Baviere (published for SCI by Elsevier Applied Science, London and New York, 1991). For example, in an enhanced oil recovery method, the displacing of the unrefined petroleum in contact with the solid material is accomplished by contacting the unrefined with a compound provided herein, wherein the unrefined petroleum is in contact with the solid material. The unrefined petroleum may be in an oil reservoir. The compound or composition provided herein can be pumped into the reservoir in accordance with known enhanced oil recovery parameters. The compound can be pumped into the reservoir as part of the aqueous compositions provided herein and, upon contacting the unrefined petroleum, form an emulsion composition provided herein.

In some embodiments, the natural solid material can be rock or regolith. The natural solid material can be a geological formation such as clastics or carbonates. The natural solid material can be either consolidated or unconsolidated material or mixtures thereof. The hydrocarbon material may be trapped or confined by “bedrock” above or below the natural solid material. The hydrocarbon material may be found in fractured bedrock or porous natural solid material. In other embodiments, the regolith is soil.

In some embodiments, an emulsion forms after the contacting step. The emulsion thus formed can be the emulsion described above. In some embodiments, the emulsion thus formed can be a microemulsion. In some embodiments, the method includes allowing an unrefined petroleum acid within the unrefined petroleum material to enter into the emulsion, thereby converting the unrefined petroleum acid into a surfactant. In other words, where the unrefined petroleum acid converts into a surfactant it is mobilized and therefore separates from the solid material.

In another aspect, a method of converting (e.g., mobilizing) an unrefined petroleum acid into a surfactant is provided. The method includes contacting a petroleum material with an aqueous composition thereby forming an emulsion in contact with the petroleum material, wherein the aqueous composition includes the compound described herein. Thus, in some embodiments, the aqueous composition is the aqueous composition described above. An unrefined petroleum acid within the unrefined petroleum material is allowed to enter into the emulsion, thereby converting the unrefined petroleum acid into a surfactant.

In some embodiments, the reactive petroleum material is in a petroleum reservoir. In some embodiments, as described above and as is generally known in the art, the unrefined petroleum acid is a naphthenic acid. In some embodiments, as described above and as is generally known in the art, the unrefined petroleum acid is a mixture of naphthenic acid. In some embodiments, the aqueous composition further includes an alkali agent.

In these embodiments, the composition can comprise a compound described herein, an alkali agent, an additional surfactant, a co-solvent, and a polymer. Methods can comprise injecting a composition of this type into a hydrocarbon reservoir comprising unrefined petroleum material in contact with the solid material. In certain embodiments, the unrefined petroleum material can comprise an active oil. In these embodiments, the composition can have a pH effective to convert unrefined petroleum acid present in the unrefined petroleum material into a surfactant.

In these embodiments, the composition can comprise a compound described herein, an alkali agent, co-solvent, and a polymer. Methods can comprise injecting a composition of this type into a hydrocarbon reservoir comprising unrefined petroleum material in contact with the solid material. In certain embodiments, the unrefined petroleum material can comprise an active oil. In these embodiments, the composition can have a pH effective to convert unrefined petroleum acid present in the unrefined petroleum material into a surfactant.

In these embodiments, the composition can comprise a compound described herein, a co-solvent, and a polymer. Methods can comprise injecting a composition of this type into a hydrocarbon reservoir comprising unrefined petroleum material in contact with the solid material.

In these embodiments, the composition can comprise a compound described herein. Methods can comprise injecting a composition of this type into a hydrocarbon reservoir comprising unrefined petroleum material in contact with the solid material. In certain embodiments, the unrefined petroleum material can comprise an active oil. In these embodiments, the composition can have a pH effective to convert unrefined petroleum acid present in the unrefined petroleum material into a surfactant.

In another aspect, a method of making a compound as described herein is provided. The methods can include contacting a suitable alcohol precursor for the co-solvent with a propylene oxide thereby forming a alkoxylate hydrophobe.

By way of non-limiting illustration, examples of certain embodiments of the present disclosure are given below.

### EXAMPLES

### Example 1: Surfactants and Surface Active Solvents (SAS) with Ultra-Short Hydrophobes

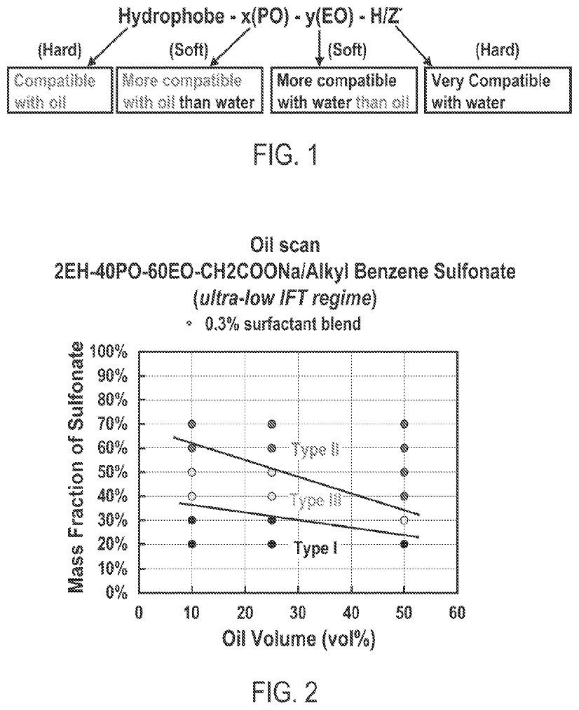

Surfactant structures can be generally represented as shown in FIG. 1. Described herein are surfactants and surface active solvents (SAS) with ultra-short hydrophobes can based on methanol, phenol, or 2 ethylhexanol, and blocks of propoxylate, ethoxylate, or any combination thereof. Optionally, an anionic end group such as a sulfate or carboxylate group. For example, the surfactant can be a MeO-xPO-yEO-H (or sulfate/carboxylate), PhO-xPO-yEO-H (or sulfate/carboxylate), or 2EHO-xPO-yEO-H (or sulfate/carboxylate). For example, co-solvent can be a MeO-xPO-yEO, PhO-xPO-yEO, or 2 EHO-xPO-yEO. To obtain surfactant behavior the number of moles of PO is greater than 10 (x>10). To obtain solvent or co-solvent behavior the number of moles of PO is less than 10 (x<10). FIGS. 2 and 3 show oil scans for tests performed using 0.3% surfactant blend of either 2-ethylhexanol(2EH)-40PO-60EO-CH2COONa with alkyl benzene sulfonate (ABS) as co-surfactant (FIG. 2) or 2EO-40PO-40EO-H with alkyl benzene sulfonate as co-surfactant (FIG. 3). The reservoir properties for the test shown in FIGS. 1 and 2 were as follows: API gravity of 38-40° (light oil), reservoir type was sandstone, reservoir temperature was 90° C., and the injection water was seawater (36,000 ppm) (hard brine). FIGS. 1 and 2 demonstrate that both formulations showed ultra-low IFT regime.

### Example 2: Surfactant and Co-Solvent Formulations for Unconventional EOR

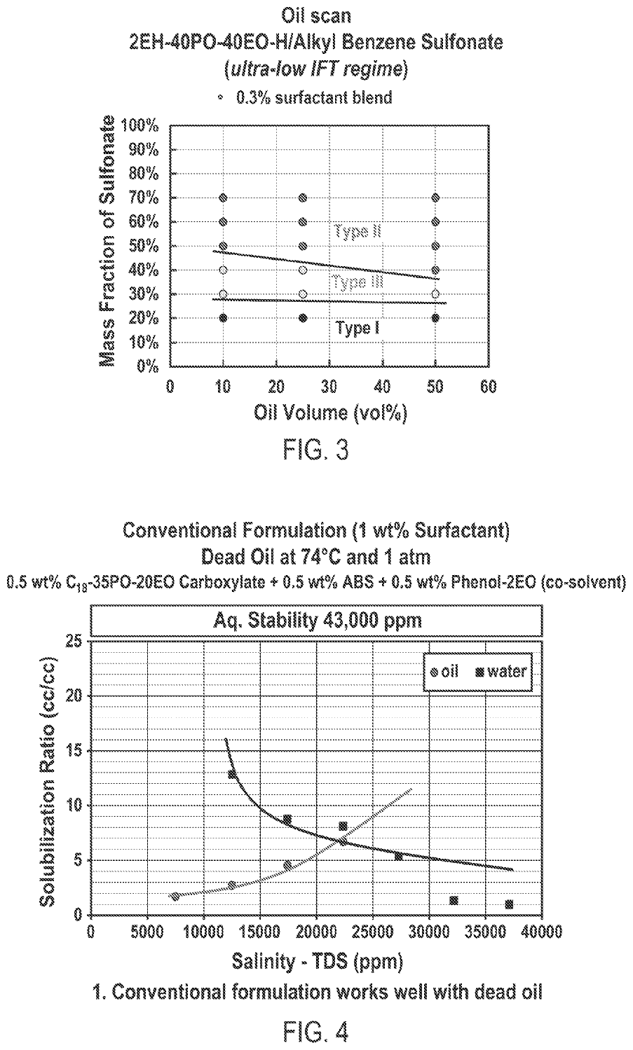

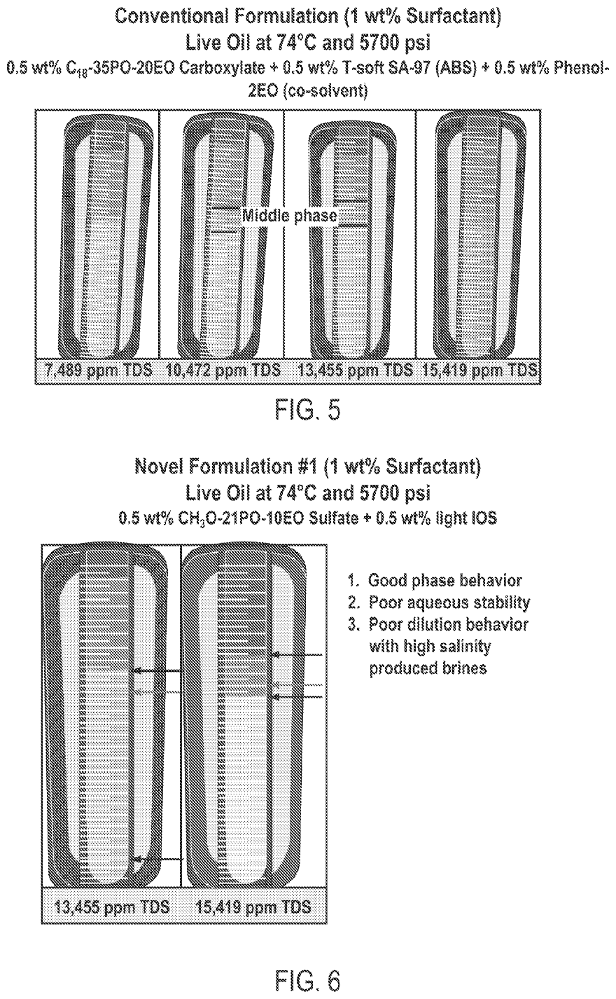

Conventional formulations work well with dead oil, but does not form ultra-low IFT with live oil (high GOR=˜3000 scf/bbl) (FIGS. 4 and 5). Accordingly, surfactants and co-solvents were studied in high-performance/low-cost formulations for unconventional EOR. Ultra short hydrophobe based surfactants (methyl based surfactants) are unique since hydrophobicity comes from the PO block. As with alkoxylates surfactants there is freedom to change number of PO and EO chain length to match the requirement of the oil and brine system. CH₃O xPO xEO (non-ionic or sulfate) surfactant shows good phase behavior with live oil but not as good with dead oil. FIG. 6 shows the phase behavior of a mixture of 0.5 wt % CH₃O-21PO-10EO sulfate and co-surfactant 0.5 wt % light internal olefin sulfonate (IOS) using live oil at 74° C. and 5700 psi. The images in FIG. 6 show middle phases and better IFT compared to conventional formulations. However, even though the IFT is favorable the aqueous stability is poor specially with high salinity produced brines since IOS is not tolerant of calcium and magnesium.

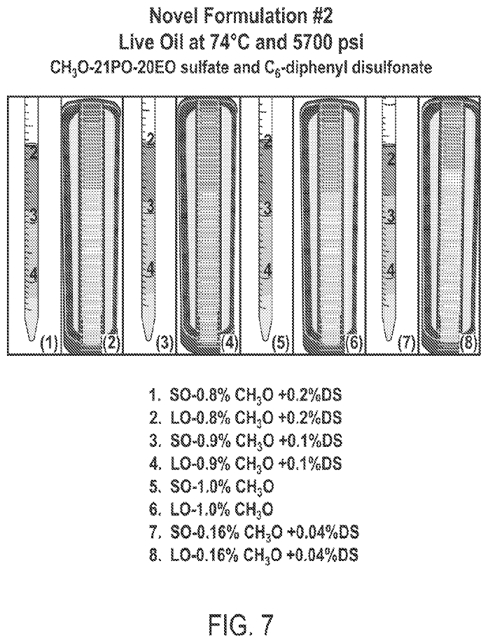

Ultra short hydrophobe based surfactants such as methyl alkoxy sulfates in combination with C6-diphenyldisulfonate (C6L) co-surfactant was used to give calcium solubility and flexibility in brines that have variable hardness/TDS. Disulfonate co-surfactant is very hydrophilic allowing for good solubility, however, interaction with the micelles is poor. HPLC analysis show that all of the disulfonate is left in the aqueous phase and none partitioned into the micelles. Different ratios of methyl alkoxy sulfates to disulfonate were tested. The results showed that although, good aqueous stability can be seen the interfacial tension (IFT) is affected by the addition of the disulfonate (FIG. 7).

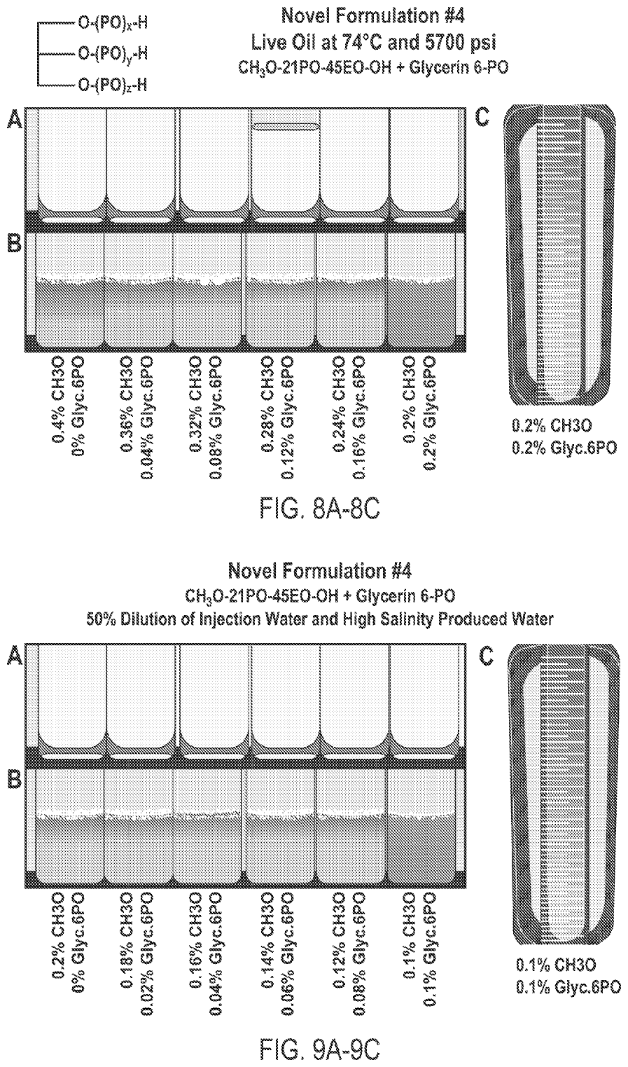

Surface-active co-solvents were identified that showed low IFT and good aqueous stability due to high tolerance of calcium in injection waters and high-salinity produced waters. Glycerine propoxylates are cheap and flexible in terms of structure. Glycerine propoxylates are very hydrophilic and tolerate calcium well specially if PO can be long enough it can show hydrophobicity and help produce IFT. Ultra short hydrophobe based surfactants were tested in combination with glycerin-xPO (glycerin-6PO) co-solvents for use in both dead oil and live oil. FIG. 8 show results for aqueous stability (FIG. 8A) and phase behavior in dead oil (FIG. 8B) and live oil (FIG. 8C) at different ratios of non-ionic CH₃O—21PO-45EO surfactant in combination with glycerin-6PO co-solvent. Results show good aqueous stability and phase behavior across all the ratios tested. IFT was especially good for surfactant to co-solvent ratio of 1:1.

FIG. 9 show results for aqueous stability (FIG. 9A) and phase behavior in dead oil (FIG. 9B) and live oil (FIG. 9C) at different ratios of non-ionic CH₃O—21PO-45EO surfactant in combination with glycerin-6PO co-solvent in high-salinity produced waters when the surfactant concentration was diluted 50% compared to surfactant concentration tested in FIG. 8. Results show good aqueous stability in high-salinity produced waters (FIG. 9A) and good phase behavior in dead oil (FIG. 9B) and live oil (FIG. 9C). IFT was good especially where the surfactant to co-solvent ratio was 1:1.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A compound defined by Formula I 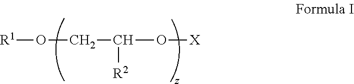
wherein
R¹ is a C₁-C₈ alkyl group or C₆ aryl group, the C₆-aryl group optionally substituted with one or two C₁-C₂ alkyl groups;
R² is, independently for each occurrence, hydrogen or methyl;
z is an integer from 10 to 75;
X is —H, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH; and
M⁺ is a cation.

2. The compound of claim 1, wherein z is from 15 to 50.

3. The compound of claim 1, wherein R¹ is a linear or branched C₁-C₆ alkyl group, such as a linear or branched C₁-C₅ alkyl group, or a linear or branched C₁-C₄ alkyl group.

4. The compound of claim 1, wherein R¹ is a C₁-C₃ alkyl group.

5. The compound of claim 1, wherein R¹ is methyl, ethyl, or phenyl.

6-7. (canceled)

8. The compound of claim 7, wherein R¹ is substituted with one C₁-C₂ alkyl group.

9. The compound of claim 7, wherein R¹ is substituted with two C₁-C₂ alkyl groups.

10. The compound of claim 1, wherein the compound is defined by Formula II 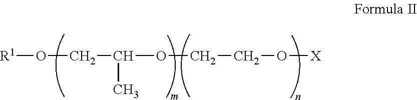
wherein
R¹ is a C₁-C₈ alkyl group or C₆ aryl group;
m is an integer from 1 to 50 and n is an integer from 1 to 50, with the proviso that m+n is from 10 to 75;
X is —H, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH; and
M⁺ is a cation.

11. The compound of claim 10, wherein m+n is from 15 to 50.

12. The compound of claim 10, wherein R¹ is a linear or branched C₁-C₆ alkyl group, such as a linear or branched C₁-C₅ alkyl group, or a linear or branched C₁-C₄ alkyl group.

13. The compound of claim 10, wherein R¹ is a C₁-C₃ alkyl group.

14. The compound of claim 10, wherein R¹ is methyl, ethyl, or phenyl.

15-16. (canceled)

17. The compound of claim 16, wherein R¹ is substituted with one C₁-C₂ alkyl group.

18. The compound of claim 16, wherein R¹ is substituted with two C₁-C₂ alkyl groups.

19. The compound of claim 10, wherein m is from 5 to 25, such as from 5 to 20 or from 10 to 20.

20. The compound of claim 10, wherein n is from 5 to 35, such as from 5 to 30 or from 10 to 25.

21. An aqueous composition comprising water and the compound of claim 1.

22-31. (canceled)

32. An emulsion comprising the the compound of claim and (ii) unrefined petroleum.

33-34. (canceled)

35. A method of displacing an unrefined petroleum material in contact with a solid material, said method comprising:
(i) contacting the unrefined petroleum material with the compound of claim 1, wherein the unrefined petroleum material is in contact with the solid material; and
(ii) allowing the unrefined petroleum material to separate from the solid material, thereby displacing the unrefined petroleum material in contact with the solid material.

36-37. (canceled)

38. A method for recovering hydrocarbons from a subterranean formation, the method comprising
(a) introducing the compound of claim 1 through a wellbore into the subterranean formation; and
(b) producing fluids from the subterranean formation;
wherein the hydrocarbons in the subterranean formation comprise live oil.

39-41. (canceled)
